(19) United States
(12) Patent Application Publication
(10) Pub. No.: US 2023/0245717 A1
(43) Pub. Date: Aug. 3, 2023

(54) INDEL PATHOGENICITY DETERMINATION

(71) Applicants: Illumina, Inc., San Diego, CA (US); Illumina Software, Inc., San Diego, CA (US)

(72) Inventors: Jeremy Francis McRae, Hayward, CA (US); Yanshen Yang, Southborough, MA (US); Marc Fasnacht, Cupertino, CA (US); Kai-How Farh, Hillsborough, CA (US)

(21) Appl. No.: 18/160,566

(22) Filed: Jan. 27, 2023

Related U.S. Application Data
(60) Provisional application No. 63/304,308, filed on Jan. 28, 2022.

(51) Int. Cl. G16B 20/50; G16B 40/00

(52) U.S. Cl. CPC G16B 20/50; G16B 40/00

(57) ABSTRACT

Described herein are technologies for converting context of an ANN or context of another type of computing system that is trainable through machine learning. In some implementations, the technologies convert a first context of a computing system (such as an ANN), which is to provide pathogenicity of variants of genomes of a population, to a second context of the computing system, which is to provide pathogenicity of indels of the genomes of the population.

### CROSS-REFERENCE TO RELATED APPLICATIONS

This application claims the benefit of, and priority to, U.S. Provisional Application No. 63/304,308, entitled “INDEL PATHOGENICITY DETERMINATION,” filed Jan. 28, 2022. The aforementioned application is hereby incorporated by reference in its entirety.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using techniques for converting context of an artificial neural network (ANN) or another type of computing system that is trainable through machine learning.

### INCORPORATIONS

The following are incorporated by reference for all purposes as if fully set forth herein:
U.S. Provisional Patent Application No. 63/253,122, titled “PROTEIN STRUCTURE-BASED PROTEIN LANGUAGE MODELS,” filed Oct. 6, 2021 (Attorney Docket No. ILLM 1050-1/IP-2164-PRV);
U.S. Provisional Patent Application No. 63/281,579, titled “PREDICTING VARIANT PATHOGENICITY FROM EVOLUTIONARY CONSERVATION USING THREE-DIMENSIONAL (3D) PROTEIN STRUCTURE VOXELS,” filed Nov. 19, 2021 (Attorney Docket No. ILLM 1060-1/IP-2270-PRV);
U.S. Provisional Patent Application No. 63/281,592, titled “COMBINED AND TRANSFER LEARNING OF A VARIANT PATHOGENICITY PREDICTOR USING GAPED AND NON-GAPED PROTEIN SAMPLES,” filed Nov. 19, 2021 (Attorney Docket No. ILLM 1061-1/IP-2271-PRV);
U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);
U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);
U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);
U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);
U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);
U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);
U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US);
U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US);
U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US);
U.S. Patent Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-1/IP-2142-PRV);
Sundaram, L. et al. Predicting the clinical impact of human mutation with deep neural networks. Nat. Genet. 50, 1161-1170 (2018) (hereinafter “PrimateAI”);
Jaganathan, K. et al. Predicting splicing from primary sequence with deep learning. Cell 176, 535-548 (2019);
U.S. Patent Application No. 63/175,767, titled “EFFICIENT VOXELIZATION FOR DEEP LEARNING,” filed on Apr. 16, 2021, (Atty. Docket No. ILLM 1048-1/IP-2143-PRV); and
U.S. patent application Ser. No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

### Neural Networks

FIG. 1 shows one implementation of an artificial neural network (ANN) with multiple layers. An ANN (or also described herein a neural network) is a system of interconnected artificial neurons (e.g., a₁, a₂, a₃) that exchange messages between each other. The illustrated neural network has three inputs, two neurons in the hidden layer and two neurons in the output layer. The hidden layer has an activation function ƒ(•) and the output layer has an activation function g(•). The connections have numeric weights (e.g., w₁₁, w₂₁, w₁₂, w₃₁, w₂₂, w₃₂, v₁₁, v₂₂) that are tuned during the training process, so that a properly trained network responds correctly when fed an image to recognize. The input layer processes the raw input, the hidden layer processes the output from the input layer based on the weights of the connections between the input layer and the hidden layer. The output layer takes the output from the hidden layer and processes it based on the weights of the connections between the hidden layer and the output layer. The network includes multiple layers of feature-detecting neurons. Each layer has many neurons that respond to different combinations of inputs from the previous layers. These layers are constructed so that the first layer detects a set of primitive patterns in the input image data, the second layer detects patterns of patterns and the third layer detects patterns of those patterns.

### SUMMARY

Described herein are technologies for converting context of an ANN or context of another type of computing system that is trainable through machine learning. In some implementations, the technologies convert a first context of a computing system (such as an ANN), which is to provide pathogenicity of variants (e.g., missense variants) of genomes of a population, to a second context of the computing system, which is to provide pathogenicity of indels of the genomes of the population.

In providing such technologies, the systems and methods described herein overcome some technical problems in obtaining scores from a computing system in which the context of the computing system is changed. Also, the techniques disclosed herein provide specific technical solutions to at least overcome the technical problems mentioned herein as well as other technical problems not described herein but recognized by those skilled in the art.

With respect to some implementations, disclosed herein are computerized methods for converting context of an ANN or context of another type of computing system, as well as a non-transitory computer-readable storage medium for carrying out technical operations of the computerized methods. The non-transitory computer-readable storage medium has tangibly stored thereon, or tangibly encoded thereon, computer readable instructions that when executed by one or more devices (e.g., one or more personal computers or servers) cause at least one processor to perform a method for converting context of an ANN or context of another type of computing system.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings.

FIG. 1 shows one implementation of a feed-forward neural network with multiple layers, which is a type of ANN.

FIG. 2 depicts a method for converting context of an artificial neural network (ANN) or context of another type of computing system that is trainable through machine learning, in accordance with some implementations of the present disclosure.

FIGS. 3 and 4 depict respective methods for converting context of an ANN or context of another type of computing system that is trainable through machine learning, in accordance with some implementations of the present disclosure. Specifically, each of FIGS. 3 and 4 depict converting a first context of a computing system, which is to provide pathogenicity of variants (e.g., missense variants) of genomes of a population, to a second context of the system, which is to provide pathogenicity of indels of the genomes of the population.

FIGS. 5, 6, and 7 depict methods that each can be part of the method shown in FIG. 4, in accordance with some implementations of the present disclosure.

FIG. 8 depicts two operations that can be combined with the method shown in FIG. 3 or the method shown in FIG. 4, in accordance with some implementations of the present disclosure.

FIG. 9 depicts a method for converting context of an ANN, specifically, in accordance with some implementations of the present disclosure. Also, FIG. 9 depicts converting a first context of the ANN, which is to provide pathogenicity of variants of genomes of a population, to a second context of the ANN, which is to provide pathogenicity of indels of the genomes of the population.

FIG. 10 depicts a block diagram of example aspects of a computing system, in accordance with some implementations of the present disclosure.

FIG. 11 depicts a plot in a two-dimensional graph showing the relationship between binned PrimateAI scores for variants and insertion variants versus natural depletion (i.e., being more depleted indicates stronger selection (i.e., propensity of a variant or insertion in genomes of a population)). In FIG. 11, natural depletion values (or the propensity values) are represented with the y-axis. And, the bins of PrimateAI scores are represented with the x-axis.

FIG. 12 depicts a scatterplot in a two-dimensional graph showing the relationship between binned PrimateAI scores for variants, insertion variants, and deletion variants versus proportions of observed variants (i.e., propensity of a variant or an indel in genomes of a population). In FIG. 12, proportions of observed variants are represented with the y-axis. And, the bins of PrimateAI scores are represented with the x-axis.

FIGS. 13 and 14 depict respective scatterplots in respective two-dimensional graphs, each plot showing the relationship between binned PrimateAI scores for variants, insertion variants, and deletion variants versus adjusted proportions of observed variants (i.e., propensity of a variant or indel in genomes of a population). In FIGS. 13 and 14, adjusted proportions of observed variants (or the adjusted ratios) are represented with the y-axis. And, the bins of PrimateAI scores are represented with the x-axis. Specifically, FIG. 13 relates to variants occurring in a three base pair in-frame in exomes. Specifically, FIG. 14 relates to variants occurring in a six base pair in-frame in exomes.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

### PrimateAI

PrimateAI is a deep residual neural network for classifying the pathogenicity of missense mutations. In at least one version, PrimateAI is trained on a dataset of ˜380,000 common variants from humans and six non-human primate species, using a semi-supervised benign vs unlabeled training regimen. In such version(s), the input to the network is the amino acid sequence flanking the variant of interest and the orthologous sequence alignments in other species, without any additional human-engineered features, and the output is the pathogenicity score from 0 (less pathogenic) to 1 (more pathogenic). In such version(s), to incorporate information about protein structure, PrimateAI can learn to predict secondary structure and solvent accessibility from amino acid sequence and includes these as sub-networks in the full model. Also, in such version(s), the total size of the network, with protein structure included, is 36 layers of convolutions, including roughly 400,000 trainable parameters.

gnomAD

The Genome Aggregation Database (gnomAD) is a resource developed by an international coalition of investigators, with the goal of aggregating and harmonizing both exome and genome sequencing data from a wide variety of large-scale sequencing projects and making summary data available for the wider scientific community. Multiple versions of the gnomAD have been released.

Described herein are techniques for converting context of an artificial neural network or another type of computing system that is trainable through machine learning. Examples of the techniques disclosed herein convert a first context for a computing system (such as an ANN) to a second context for the computing system. Specifically, the first context for the computing system is pathogenicity of variants (e.g., missense variants) of genomes of a population, and the second context for the computing system is pathogenicity of indels of the genomes of the population. To put it another way, some of the techniques disclosed herein provide operations for converting a computing system or the output of the computing system, which is initially meant to provide pathogenicity of variants (e.g., missense variants) of genomes of a population, to a computing system or the output of the computing system that provides pathogenicity of indels of the genomes of the population.

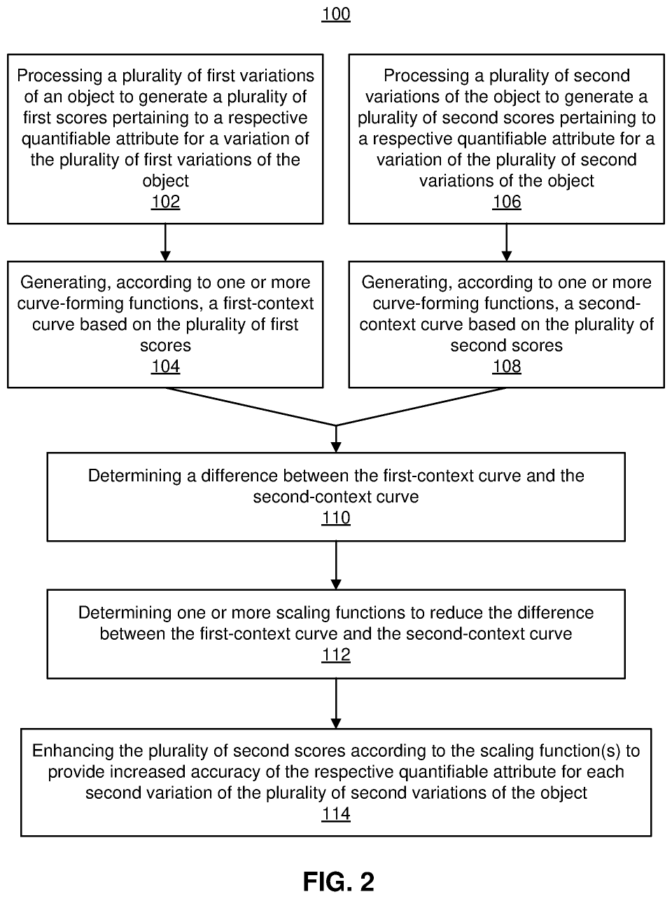

The actions of FIGS. 2-9 can be implemented at least partially with and/or by one or more processors configured to receive or retrieve information, process the information, store results, and transmit the results. Other implementations may perform the actions in different orders and/or with different, fewer, or additional actions than those illustrated in FIGS. 2-9. Multiple actions can be combined in some implementations. For convenience, this figure is described with reference to the system that carries out a method. The system is not necessarily part of the method. The actions of FIGS. 2-9 can be executed in parallel or in sequence.

FIG. 2 illustrates a method 100 that converts a first context for a computing system (such as an ANN) to a second context for the computing system.

In one implementation, the ANN is a multilayer perceptron (MLP). In another implementation, the ANN is a feedforward neural network. In yet another implementation, the ANN is a fully-connected neural network. In a further implementation, the ANN is a fully convolution neural network. In yet further implementation, the ANN is a semantic segmentation neural network. In yet another further implementation, the ANN is a generative adversarial network (GAN) (e.g., CycleGAN, StyleGAN, pixelRNN, text-2-image, DiscoGAN, IsGAN).

In one implementation, the ANN is a convolution neural network (CNN) with a plurality of convolution layers. In another implementation, the ANN is a recurrent neural network (RNN) such as a long short-term memory network (LSTM), bi-directional LSTM (Bi-LSTM), or a gated recurrent unit (GRU). In yet another implementation, the ANN includes both a CNN and an RNN.

In yet other implementations, the ANN can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The ANN can use one or more loss functions such as logistic regression/log loss, multi-class cross-entropy/softmax loss, binary cross-entropy loss, mean-squared error loss, L1 loss, L2 loss, smooth L1 loss, and Huber loss. The ANN can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The ANN can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential linear unit (ELU), sigmoid and hyperbolic tangent (tanh)), batch normalization layers, regularization layers, dropout, pooling layers (e.g., max or average pooling), global average pooling layers, and attention mechanisms (e.g., self-attention).

The ANN can be a rule-based model, linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The ANN can be an ensemble of multiple models, in some implementations.

The ANN is trained using backpropagation-based gradient update techniques. Example gradient descent techniques that can be used for training the ANN include stochastic gradient descent, batch gradient descent, and mini-batch gradient descent. Some examples of gradient descent optimization algorithms that can be used to train the ANN are Momentum, Nesterov accelerated gradient, Adagrad, Adadelta, RMSprop, Adam, AdaMax, Nadam, and AMSGrad.

In different implementations, the ANN includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, RoBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

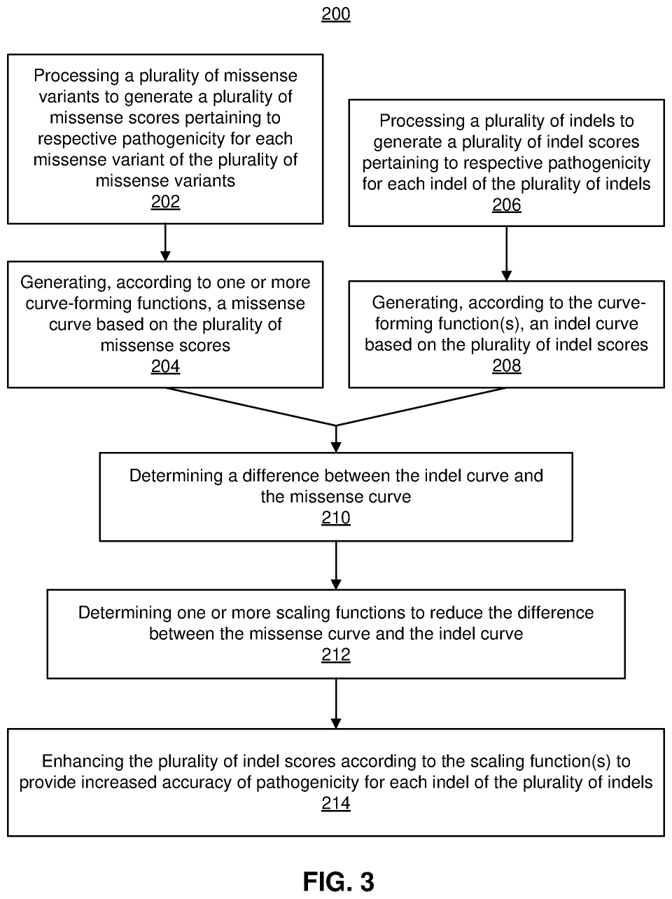

FIG. 3 illustrates a method 200 that converts a first context of a computing system context, which is to provide pathogenicity of variants (e.g., missense variants) of genomes of a population, to a second context of providing pathogenicity of indels of the genomes of the population.

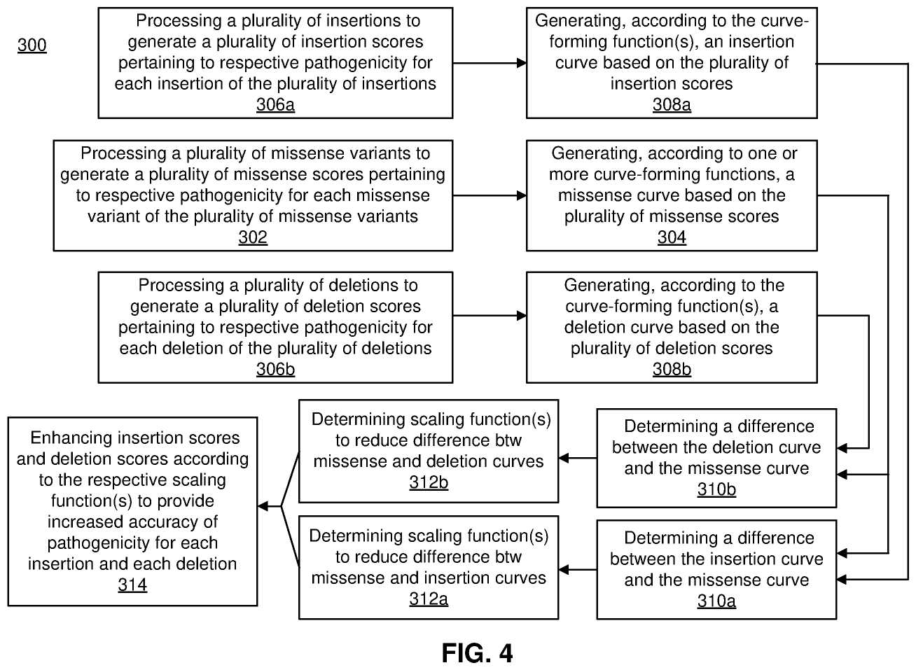

FIG. 4 illustrates a method 300 that converts a first context of a computing system context, which is to provide pathogenicity of variants (e.g., missense variants) of genomes of a population, to a second context of providing pathogenicity of indels of the genomes of the population. Although, as shown in FIG. 4, the plurality of indels specifically includes a plurality of insertions and a plurality of deletions of the genomes of the population.

For the purpose of this disclosure, it is to be understood that a plurality of indels, in general, includes a plurality of insertions and/or a plurality of deletions. Also, for the purpose of this disclosure, it is to be understood that a variant in a generic term for a variant or an indel variant (i.e., an indel). And, for the purpose of this disclosure, it is to be understood that an indel variant (i.e., an indel) is a generic term for an insertion variant (i.e., an insertion) or a deletion variant (i.e., a deletion). And, unless specified otherwise herein, the term “variant” refers to a nucleic acid sequence that is different from a nucleic acid reference. Typical nucleic acid sequence variant includes without limitation single nucleotide polymorphism (SNP), short deletion and insertion polymorphisms (indel), copy number variation (CNV), microsatellite markers or short tandem repeats and structural variation. Somatic variant calling is the effort to identify variants present at low frequency in the DNA sample. Somatic variant calling is of interest in the context of cancer treatment. Cancer is caused by an accumulation of mutations in DNA. A DNA sample from a tumor is generally heterogeneous, including some normal cells, some cells at an early stage of cancer progression (with fewer mutations), and some late-stage cells (with more mutations). Because of this heterogeneity, when sequencing a tumor (e.g., from an FFPE sample), somatic mutations will often appear at a low frequency. For example, a SNV might be seen in only 10% of the reads covering a given base. A variant that is to be classified as somatic or germline by the variant classifier is also referred to herein as the “variant under test.”

Method 100 commences with step 102, which includes processing a plurality of first variations of an object to generate a plurality of first scores pertaining to a respective quantifiable attribute for a variation of the plurality of first variations of the object. Method 100 then continues with step 104, which includes generating, according to one or more curve-forming functions, a first-context curve based on the plurality of first scores.

“Function” or “logic” (e.g., curve-forming functions), as used herein, can be implemented in the form of a computer product including a non-transitory computer readable storage medium with computer usable program code for performing the method steps described herein. The “logic” can be implemented in the form of an apparatus including a memory and at least one processor that is coupled to the memory and operative to perform exemplary method steps. The “logic” can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media). In one implementation, the logic implements a data processing function. The logic can be a general purpose, single core or multicore, processor with a computer program specifying the function, a digital signal processor with a computer program, configurable logic such as an FPGA with a configuration file, a special purpose circuit such as a state machine, or any combination of these. Also, a computer program product can embody the computer program and configuration file portions of the logic.

Also, the method 100 commences with step 106, which includes processing a plurality of second variations of the object to generate a plurality of second scores pertaining to a respective quantifiable attribute for a variation of the plurality of second variations of the object. Method 100 then continues with step 108, which includes generating, according to one or more curve-forming functions, a second-context curve based on the plurality of second scores.

Next, the method 100 continues with step 110, which includes determining selection pattern differences between the first-context curve and the second-context curve. Then, the method 100 continues with step 112, which includes determining one or more scaling functions to reduce the selection pattern differences between the first-context curve and the second-context curve. Finally, at step 114, the method 100 continues with enhancing/calibrating/recalibrating/updating/optimizing/modifying the plurality of second scores according to the scaling function(s) to provide increased accuracy of the respective quantifiable attribute for each second variation of the plurality of second variations of the object.

In some implementations of the method 100 (such as method 200 shown in FIG. 3), the plurality of first variations of an object is a plurality of variants of genomes of a population and the plurality of second variations of the object is a plurality of indels of the genomes. Also, in such implementation of the method 100, the plurality of first scores is a plurality of missense pathogenicity scores for each variant of the plurality of variants and the plurality of second scores is a plurality of indel pathogenicity scores for each indel of the plurality of indels. Also, in such implementations of the method 100, the first-context curve is a missense curve based on the plurality of missense pathogenicity scores and the second-context curve is an indel curve based on the plurality of indel pathogenicity scores.

Method 200 commences with step 202, which includes processing a plurality of variants to generate a plurality of missense pathogenicity scores for each variant of the plurality of variants. Method 200 then continues with step 204, which includes generating, according to one or more curve-forming functions, a missense curve based on the plurality of missense pathogenicity scores.

Also, the method 200 commences with step 206, which includes processing a plurality of indels to generate a plurality of indel pathogenicity scores for each indel of the plurality of indels. Method 200 then continues with step 208, which includes generating, according to the curve-forming function(s), an indel curve based on the plurality of indel pathogenicity scores.

Next, the method 200 continues with step 210, which includes determining selection pattern differences between the indel curve and the missense curve. Then, the method 200 continues with step 212, which includes determining one or more scaling functions to reduce the selection pattern differences between the missense curve and the indel curve. Finally, at step 214, the method 200 continues with enhancing/calibrating/recalibrating/updating/optimizing/modifying the plurality of indel pathogenicity scores according to the scaling function(s) to provide a recalibrated accuracy of indel pathogenicity score for each indel of the plurality of indels.

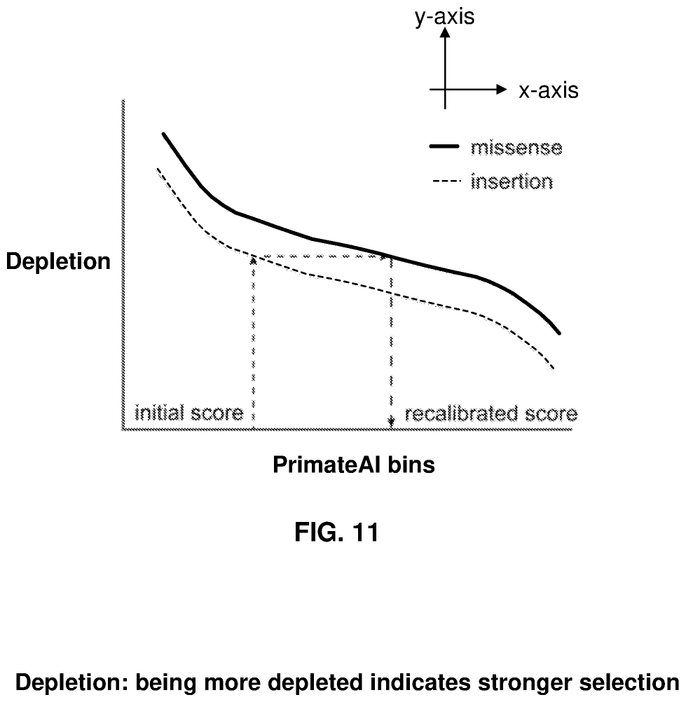

In some implementations of the aforesaid methods, the curve-forming function(s) include a function that accounts for proportions of different indels and proportions of different variants in genomes of a population. For instance, in some implementations, the curve-forming function(s) include a function that accounts for natural selection of different indels and natural selection of different variants in the genomes of the population. See FIG. 11 for an example of results of a function that accounts for natural selection of such variants.

In more generic implementations, the curve-forming function(s) include a function that accounts for proportions of the first variations of the object and proportions of the second variations of the object in populations of the object.

In some implementations of the aforesaid methods (such as method 300 shown in FIG. 4), the plurality of indels includes a plurality of insertions and a plurality of deletions, and wherein the plurality of indel pathogenicity scores includes a plurality of insertion scores and a plurality of deletion scores, respectively.

Method 300 commences with step 302, which includes processing a plurality of variants to generate a plurality of missense pathogenicity scores for each variant of the plurality of variants. Method 300 then continues with step 304, which includes generating, according to one or more curve-forming functions, a missense curve based on the plurality of missense pathogenicity scores.

Also, the method 300 commences with step 306a, which includes processing a plurality of insertions to generate a plurality of insertion scores for each insertion of the plurality of insertions. The method 300 also commences with step 306b, which includes processing a plurality of deletions to generate a plurality of deletion scores for each deletion of the plurality of deletions. Method 300 then continues with step 308a, which includes generating, according to the curve-forming function(s), an insertion curve based on the plurality of insertion scores. Also, method 300 continues with step 308b, which includes generating, according to the curve-forming function(s), a deletion curve based on the plurality of deletion scores.

Next, the method 300 continues with step 310a, which includes determining selection pattern differences between the insertion curve and the missense curve. Also, the method 300 continues with step 310b, which includes determining selection pattern differences between the deletion curve and the missense curve. Then, the method 300 continues with step 312a, which includes determining one or more scaling functions to reduce the selection pattern differences between the missense curve and the insertion curve. Further, the method 300 continues with step 312b, which includes determining additional one or more scaling functions to reduce the selection pattern differences between the missense curve and the deletion curve. Finally, at step 314, the method 300 continues with enhancing/calibrating/recalibrating/updating/optimizing/modifying the plurality of insertion scores and the plurality of deletion scores according to the respective scaling function(s) to provide a recalibrated accuracy of insertion pathogenicity score for each insertion of the plurality of insertions and each deletion of the plurality of deletions.

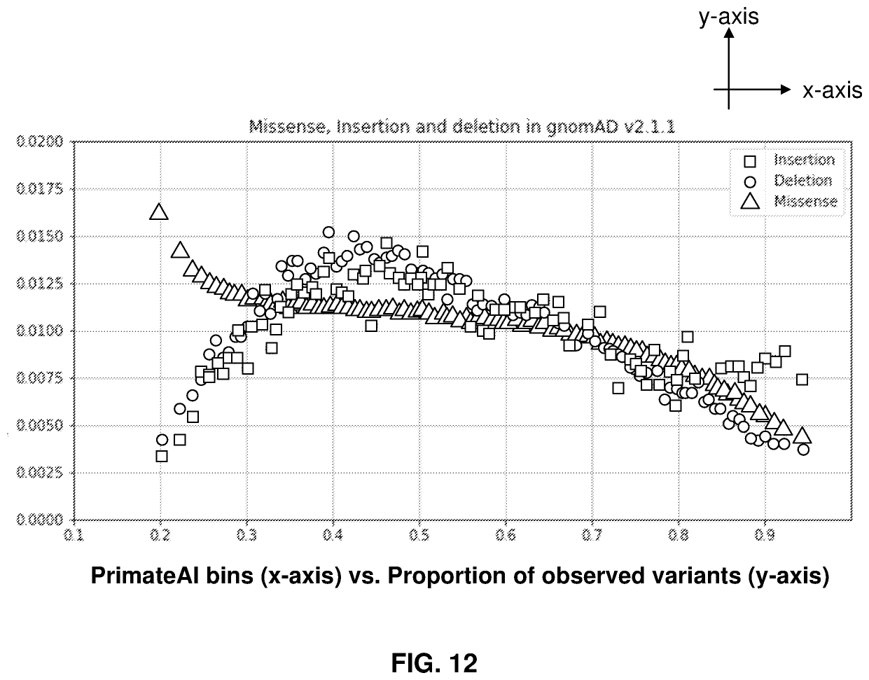

In some implementations of the aforesaid methods (e.g., see FIG. 4), the insertion curve includes a first plurality of data points including an insertion propensity score for each bin of a group of bins. Also, in such implementations, the deletion curve includes a second plurality of data points including a deletion propensity score for each bin of the group of bins. And, in such examples, the missense curve includes a third plurality of data points including a missense propensity score for each bin of the group of bins. For an example of such data points being displayed on a graph, see FIGS. 12 to 14.

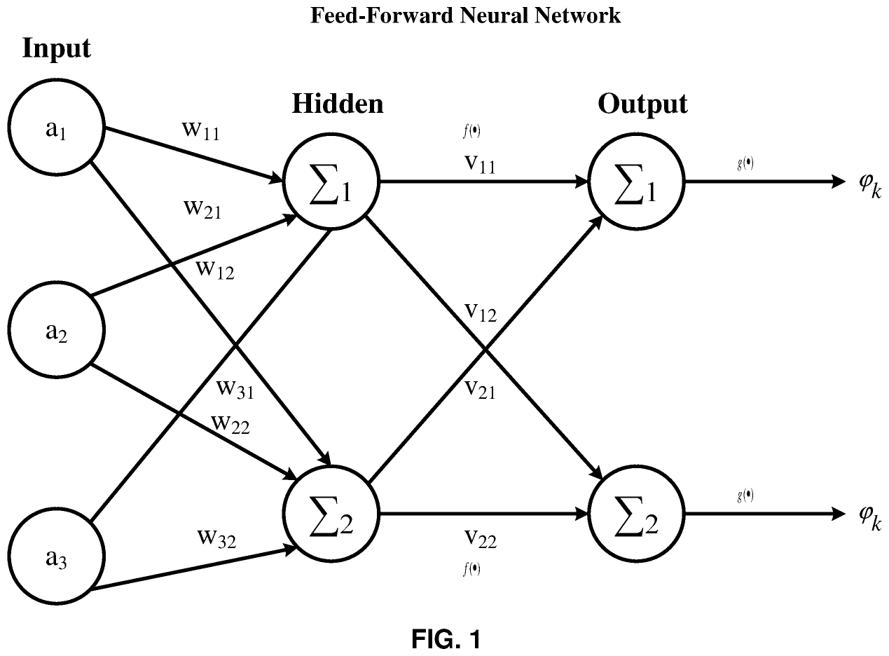

In more generic examples (e.g., see FIG. 3), the indel curve includes a plurality of data points including an indel propensity score for each bin of a group of bins. And, in such more generic examples, the missense curve includes a plurality of data points including a missense propensity score for each bin of the group of bins. In even more generic examples (e.g., see FIG. 1), the first-context curve includes a plurality of data points including a first-context propensity score for each bin of a group of bins. And, in such even more generic examples, the second-context curve includes a plurality of data points including a second-context propensity score for each bin of the group of bins.

In some implementations of the aforesaid methods (e.g., see FIG. 4), the insertion propensity score for a bin of the group of bins relates to a proportion of different insertions in the genomes of the population that have insertion scores of the plurality of insertion scores that are associated with the bin. In such examples, the deletion propensity score for a bin of the group of bins relates to a proportion of different deletions in the genomes of the population that have deletion scores of the plurality of deletion scores that are associated with the bin, and the missense propensity score for a bin of the group of bins relates to a proportion of variants in the genomes of the population that have missense pathogenicity scores of the plurality of missense pathogenicity scores that are associated with the bin. In more generic examples (e.g., see FIG. 3), the indel propensity score for a bin of the group of bins relates to a proportion of different indels in the genomes of the population that have indel pathogenicity scores of the plurality of indel pathogenicity scores that are associated with the bin. In such more generic examples, the missense propensity score for a bin of the group of bins relates to a proportion of variants in the genomes of the population that have missense pathogenicity scores of the plurality of missense pathogenicity scores that are associated with the bin. In even more generic examples (e.g., see FIG. 1), the first-context propensity score for a bin of the group of bins relates to a proportion of different first variations of the object of the population that have first-context scores of the plurality of first-context scores that are associated with the bin. In such even more generic examples, the second-context propensity score for a bin of the group of bins relates to a proportion of different second variations of the object of the population that have second-context scores of the plurality of second-context scores that are associated with the bin.

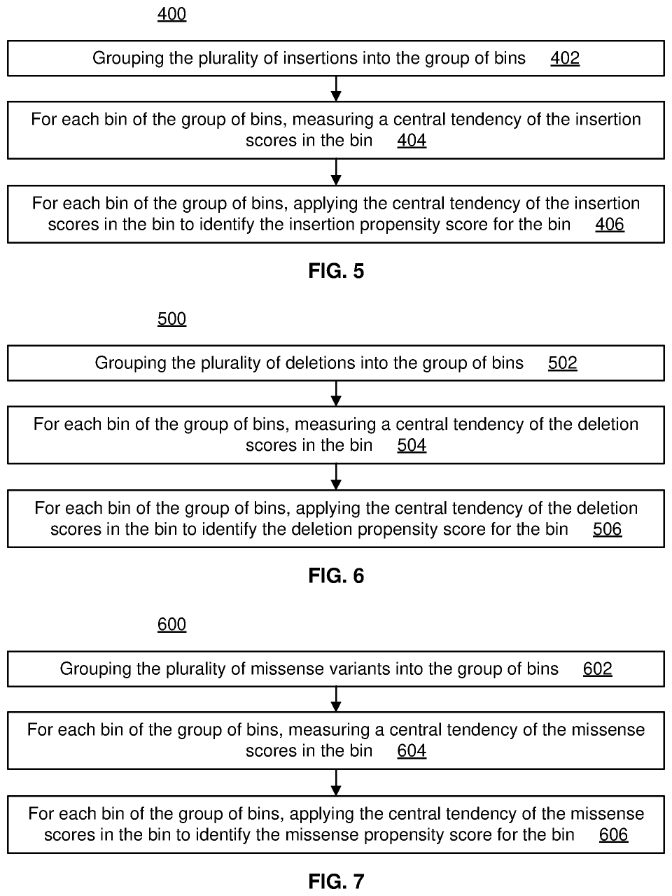

FIG. 5, illustrates a method 400 that, in some implementations, is a part of step 308a of method 300 (which includes the generation of the insertion curve). In such implementations, the generating of the insertion curve at step 308a includes grouping the plurality of insertions into the group of bins at step 402 of method 400. Also, step 308a includes step 404, which includes, for each bin of the group of bins, measuring a central tendency distribution of the insertion scores in the bin. And, step 308a also includes step 406, which includes, for each bin of the group of bins, applying the central tendency distribution of the insertion scores in the bin to identify the insertion propensity score for the bin.

FIG. 6, illustrates a method 500 that, in some implementations, is a part of step 308b of method 300 (which includes the generation of the deletion curve). In such implementations, the generating of the deletion curve at step 308b includes grouping the plurality of deletions into the group of bins at step 502 of method 500. Also, step 308b includes step 504, which includes, for each bin of the group of bins, measuring a central tendency distribution of the deletion scores in the bin. And, step 308b also includes step 506, which includes, for each bin of the group of bins, applying the central tendency distribution of the insertion scores in the bin to identify the insertion propensity score for the bin.

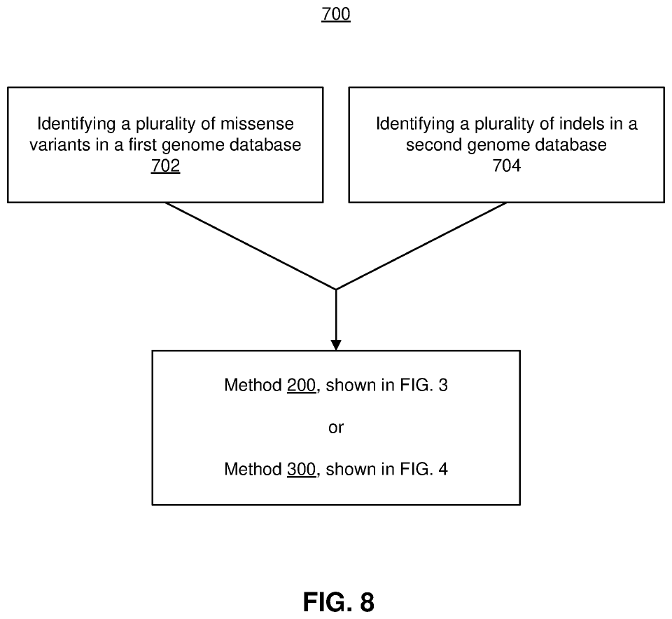

FIG. 7, illustrates a method 600 that, in some implementations, is a part of step 304 of method 300 (which includes the generation of the missense curve). In such implementations, the generating of the missense curve at step 304 includes grouping the plurality of variants into the group of bins at step 602 of method 600. Also, step 304 includes step 604, which includes, for each bin of the group of bins, measuring a central tendency distribution of the missense pathogenicity scores in the bin. And, step 304 also includes step 606, which includes, for each bin of the group of bins, applying the central tendency distribution of the missense pathogenicity scores in the bin to identify the missense propensity score for the bin.

Analogous techniques to the techniques shown in FIGS. 5 to 7 can be applied to more generic implementations using a plurality of indels and a plurality of variants. Also, analogous techniques to the techniques shown in FIGS. 5 to 7 can be applied to even more generic implementations using a plurality of first variations of an object of a population and a plurality of second variations of the object. For example, in some implementations (such as with respect to FIG. 3), the generating of the indel curve includes grouping the plurality of indels into a group of bins. Also, the generating of the missense curve includes grouping the plurality of variants into the group of bins. Also, in some examples, the generating of the indel curve includes, for each bin of the group of bins: measuring a central tendency distribution of the indel pathogenicity scores in the bin; and applying the central tendency distribution of the indel pathogenicity scores in the bin to identify an indel propensity score for the bin. Furthermore, in such examples, the generating of the missense curve includes, for each bin of the group of bins: measuring a central tendency distribution of the missense pathogenicity scores in the bin; and applying the central tendency distribution of the missense pathogenicity scores in the bin to identify a missense propensity score for the bin.

In some implementations, measuring the central tendency distribution of the indel pathogenicity scores includes determining a mean of the indel pathogenicity scores. For example, measuring the central tendency distribution of the insertion scores includes determining a mean of the insertion scores and measuring the central tendency distribution of the deletion scores includes determining a mean of the deletion scores. Also, in some implementations, measuring the central tendency distribution of the missense pathogenicity scores includes determining a mean of the missense pathogenicity scores. In some implementations, measuring the central tendency distribution of the indel pathogenicity scores includes determining a mode of the indel pathogenicity scores. For example, measuring the central tendency distribution of the insertion scores includes determining a mode of the insertion scores and measuring the central tendency distribution of the deletion scores includes determining a mode of the deletion scores. Also, in some implementations, measuring the central tendency distribution of the missense pathogenicity scores includes determining a mode of the missense pathogenicity scores. In some implementations, measuring the central tendency distribution of the indel pathogenicity scores includes determining a median of the indel pathogenicity scores. For example, measuring the central tendency distribution of the insertion scores includes determining a median of the insertion scores and measuring the central tendency distribution of the deletion scores includes determining a median of the deletion scores. Also, in some implementations, measuring the central tendency distribution of the missense pathogenicity scores includes determining a median of the missense pathogenicity scores. Also, such techniques apply to even more generic implementations as well. For example, measuring the central tendency distribution of the first-context scores includes determining a mean, mode, or median of the first-context scores. And, measuring the central tendency distribution of the second-context scores includes determining a mean, mode, or median of the second-context scores.

In some implementations of the aforesaid methods (e.g., see FIG. 4), the insertion propensity score for a bin of the group of bins represents a probability of one of the plurality of insertions associated with the bin occurs in the genomes of the population given a set of observed insertions. In such implementations, the deletion propensity score for the bin represents a probability of one of the plurality of deletions associated with the bin occurs in the genomes of the population given a set of observed deletions, and the missense propensity score for the bin represents a probability of one of the plurality of variants associated with the bin occurs in the genomes of the population given a set of observed variants. In some of the aforementioned implementations, the propensity scores reduce selection bias by equating groups based on covariates, and the covariates are the set of observed insertions, the set of observed deletions, and the set of observed variants, respectively.

In more generic examples (e.g., see FIG. 3), the indel propensity score for a bin of the group of bins represents a probability of one of the plurality of indels associated with the bin occurs in the genomes of the population given a set of observed indels. And, in such more generic implementations, the missense propensity score for the bin represents a probability of one of the plurality of variants associated with the bin occurs in the genomes of the population given a set of observed variants. Also, in some of the aforementioned implementations, the propensity scores reduce selection bias by equating groups based on covariates, and the covariates are the set of observed indels and the set of observed variants, respectively.

In even more generic examples (e.g., see FIG. 1), the first-context propensity score for a bin of the group of bins represents a probability of one of the plurality of first variations of the object associated with the bin occurs in the population given a set of observed first variations. And, in such more generic implementations, the second-context propensity score for a bin of the group of bins represents a probability of one of the plurality of second variations of the object associated with the bin occurs in the population given a set of observed second variations. Also, in some of the aforementioned implementations, the propensity scores reduce selection bias by equating groups based on covariates, and the covariates are the set of observed first variations and the set of observed second variations, respectively.

In some implementations of the aforesaid methods (e.g., see FIG. 4), the insertion curve is generated when the first plurality of data points is plotted on a two-dimensional graph with one axis for propensity scores and the other axis for the group of bins. In such examples, the deletion curve is generated when the second plurality of data points is plotted on the two-dimensional graph, and the missense curve is generated when the third plurality of data points is plotted on the two-dimensional graph. In more generic examples (e.g., see FIG. 3), the indel curve is generated when the corresponding plurality of data points for the indels is plotted on a two-dimensional graph with one axis for propensity scores and the other axis for the group of bins. In such more generic examples, the missense curve is generated when the corresponding plurality of data points for the variants is plotted on the two-dimensional graph. In even more generic examples (e.g., see FIG. 1), the first-context curve is generated when the corresponding plurality of data points for the first variations of the object is plotted on a two-dimensional graph with one axis for propensity scores and the other axis for the group of bins. In such even more generic examples, the second-context curve is generated when the corresponding plurality of data points for the second variations of the object is plotted on the two-dimensional graph. In some examples of the aforementioned implementations, the two-dimensional graph includes a set of ordered pairs (x, y), wherein f(x)=y, wherein x is the group of bins, and wherein y is the propensity scores. For an example of such data points being displayed on a graph, see FIGS. 12 to 14.

In some implementations of the aforesaid methods (e.g., see FIG. 4), the one or more scaling functions (for variants), the one or more scaling functions (for the insertions) and the one or more scaling functions (for the deletions) are part of the aforementioned scaling function(s). And, such scaling function(s) include functions to scale the proportions of different insertions, different deletions, and different variants in the genomes of the population, respectively, since indels and single-nucleotide variants have different mutability. In more generic examples (e.g., see FIG. 3), the one or more scaling functions (for variants) and the one or more scaling functions (for the indels) are part of the aforementioned scaling function(s). And, such scaling function(s) include functions to scale the proportions of different indels and different variants in the genomes of the population, respectively. In even more generic examples (e.g., see FIG. 1), the one or more scaling functions (for the first variations of the object) and the one or more scaling functions (for the second variations of the object) are part of the aforementioned scaling function(s). And, such scaling function(s) include functions to scale the proportions of different first variations of the object and different second variations of the object, respectively.

In some implementations (e.g., see FIGS. 3 and 4), the scaling function(s) obtain scaling factors from comparable variants under natural selection. See FIG. 11 for an example of results of a function that accounts for natural selection of variants. For example, in some implementations (e.g., see FIG. 4), the enhancing/calibrating/recalibrating/updating/optimizing/modifying of the plurality of insertion scores includes scaling the insertion propensity scores according to first scaling factors of the scaling factors that are associated with insertions in the genomes of the population. In such examples, the enhancing/calibrating/recalibrating/updating/optimizing/modifying of the plurality of deletion scores includes scaling the deletion propensity scores according to second scaling factors of the scaling factors that are associated with deletions in the genomes of the population, and the enhancing/calibrating/recalibrating/updating/optimizing/modifying of the plurality of missense pathogenicity scores includes scaling the missense propensity scores according to third scaling factors of the scaling factors that are associated with variants in the genomes of the population. Also, for example, in some implementations (e.g., see FIG. 3), the enhancing/calibrating/recalibrating/updating/optimizing/modifying of the plurality of indel pathogenicity scores includes scaling the indel propensity scores according to first scaling factors of the scaling factors that are associated with indels in the genomes of the population. In such examples, the enhancing/calibrating/recalibrating/updating/optimizing/modifying of the plurality of missense pathogenicity scores includes scaling the missense propensity scores according to second scaling factors of the scaling factors that are associated with variants in the genomes of the population.

In some implementations, comparable variants of the variants are synonymous mutations for variants. And, in some of such examples, the enhancing/calibrating/recalibrating/updating/optimizing/modifying of the plurality of missense pathogenicity scores includes calibrating missense propensity scores based on the synonymous mutations for variants.

In some implementations, the comparable variants of the indels are indels in coding and noncoding regions of the genomes of the population. And, in some of such examples, the enhancing/calibrating/recalibrating/updating/optimizing/modifying of the plurality of insertion scores includes calibrating insertion propensity scores based on an observed versus expected ratio based on insertions occurring in coding regions versus noncoding regions of the genomes of the population, respectively. Also, in such instances, the enhancing/calibrating/recalibrating/updating/optimizing/modifying of the plurality of deletion scores includes calibrating deletion propensity scores based on an observed versus expected ratio based on deletions occurring in coding regions versus noncoding regions of the genomes of the population, respectively.

In some implementations, the group of bins represents all the scores, and each bin of the group of bins represents a different range of scores in all the scores. Also, in some examples, all the scores includes the plurality of insertion scores, the plurality of deletion scores, and the plurality of missense pathogenicity scores. In some examples, all the scores includes the plurality of indel pathogenicity scores and the plurality of missense pathogenicity scores. And, in more even more generic examples, all the scores includes the plurality of first-context scores and the plurality of second-context scores.

In some implementations, each bin of the group of bins is associated with a certain amount of the plurality of insertions that have scores within a respective range of scores associated with the bin. Also, each bin of the group of bins is associated with a certain amount of the plurality of deletions that have scores within a respective range of scores associated with the bin. And, each bin of the group of bins is associated with a certain amount of the plurality of variants that have scores within a respective range of scores associated with the bin. The same can be said for more generic examples, with the bins being associated with indel and missense pathogenicity scores. And, the same can be said for even more generic examples, with the bins being associated with first-context scores and second-context scores.

In some implementations, the group of bins includes a group of percentile bins. For instance, the group of percentile bins includes one hundred bins. And, with the one hundred bins, a first bin of the one hundred bins represents scores that range from 0 to 0.01 and a one hundredth bin represents scores that range from 0.99 to 1. The bins between the first bin and the one hundredth bin each include a range of scores of a percentile.

In some implementations (e.g., see FIG. 4), the indel pathogenicity scores are generated by an artificial neural network (ANN), and the processing of the plurality of insertions and the plurality of deletions is implemented by the ANN. In some implementations (e.g., see FIG. 3), the indel pathogenicity scores are generated by an ANN, and the processing of the plurality of indels is implemented by the ANN. And, in some implementations (e.g., see FIG. 2), the processing of the plurality of first variations of the object is implemented by an ANN, and the processing of the plurality of second variations of the object is implemented by the ANN.

In some implementations, (e.g., see FIGS. 3 and 4), the ANN is configured to classify pathogenicity of variants. In some examples of such implementations, the ANN includes a deep residual neural network for classifying pathogenicity of missense mutations. Even more specific, in some examples, the ANN includes a version of PrimateAI.

FIG. 8, illustrates a method 700 that converts a first context of a computing system context, which is to provide pathogenicity of variants of genomes of a population, to a second context of providing pathogenicity of indels of the genomes of the population. For brevity's sake, an additional figure, similar to FIG. 8, that separates out processing steps for indels into analogous steps for insertions and deletions is not provided. Method 700 commences with step 702, which includes identifying a plurality of variants in a first genome database. Also, method 700, starts with step 704, which includes identifying a plurality of indels in a second genome database. After steps 702 and 704, the method 700 continues with the steps of method 200 or the steps of the method 300, depending on the implementation of method 700.

As mentioned herein and with respect to FIG. 8, it is to be understood that a plurality of indels, in general, includes a plurality of insertions and/or a plurality of deletions. Also, it is to be understood that FIG. 3 is a generalization of FIG. 4. In other words, FIG. 4 illustrates a more specific method that is also disclosed by FIG. 3. FIG. 3 pertains to indels, which can be insertions and/or deletions; and, FIG. 4 pertains to implementations with both insertions and deletions.

In some implementations, the first genome database includes a version of a Genome Aggregation Database (gnomAD). In some of such implementations, the second genome database includes a version of the gnomAD. In some instances, the second genome database and the first genome database are the same version of the gnomAD; and in some other implementations, the second and first genome databases are different versions of the gnomAD.

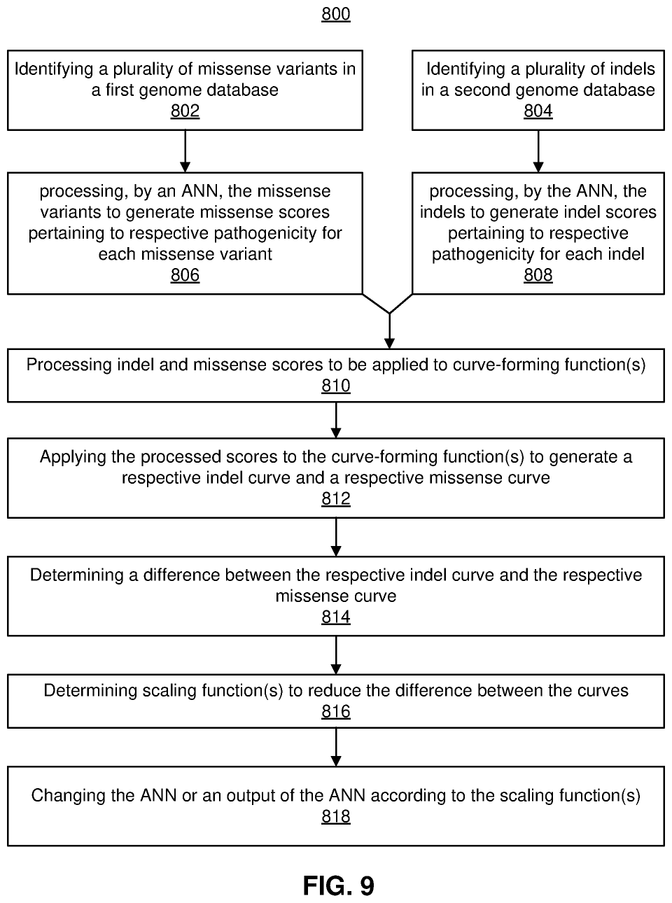

FIG. 9, illustrates a method 800 that converts a first context of a computing system context, which is to provide pathogenicity of variants of genomes of a population, to a second context of providing pathogenicity of indels of the genomes of the population. For brevity's sake, an additional figure, similar to FIG. 9, that separates out processing steps for indels into analogous steps for insertions and deletions is not provided.

Method 800 commences with step 802, which includes identifying a plurality of variants in a first genome database. Also, method 800, starts with step 804, which includes identifying a plurality of indels in a second genome database. Method 800 continues with an artificial neural network (ANN) generating a plurality of missense pathogenicity scores for each variant of a plurality of variants (at step 806). Also, method 800 continues with the ANN generating a plurality of indel pathogenicity scores for each indel of a plurality of indels (at step 808). At step 810, the method 800 continues with further processing the plurality of indel pathogenicity scores and the plurality of missense pathogenicity scores to be applied to one or more curve-forming functions. At step 812, the method 800 continues with applying the further processed scores to the curve-forming function(s) to generate an indel curve and a missense curve. At step 814, the method 800 continues with determining selection pattern differences between the indel curve and the missense curve. At step 816, the method 800 continues with determining one or more scaling functions to reduce the selection pattern differences between the curves. At step 818, the method 800 continues with updating coefficients of the ANN according to the scaling function(s). In some implementations, the updating the coefficients of the ANN according to the scaling function(s) includes enhancing/calibrating/recalibrating/updating/optimizing/modifying the plurality of indel pathogenicity scores according to the scaling function(s) to provide a recalibrated accuracy of indel pathogenicity score for each indel of the plurality of indels.

With respect to FIG. 9, in some implementations, the further processing of the plurality of indel pathogenicity scores and the plurality of missense pathogenicity scores includes: grouping the plurality of variants into a group of bins and grouping the plurality of indels into the group of bins. Also, the further processing of the plurality of indel pathogenicity scores and the plurality of missense pathogenicity scores includes, for each bin of the group of bins, measuring a central tendency distribution of the indel pathogenicity scores in the bin and measuring a central tendency distribution of the missense pathogenicity scores in the bin. The applying of the further processed scores to the curve-forming function(s) to generate the indel curve and the missense curve includes applying the central tendencies of the indel pathogenicity scores and the missense pathogenicity scores to the curve-forming function(s) to generate the indel curve and the missense curve.

With respect to FIG. 9, in some implementations, the curve-forming function(s) include a function that accounts for proportions of different indels and proportions of different variants in genomes of a population. In some of such implementations, the curve-forming function(s) include a function that accounts for natural selection of different indels and natural selection of different variants in the genomes of the population. See FIG. 11 for an example of results of a function that accounts for natural selection of such variants.

With respect to FIG. 9, in some implementations, the plurality of indels includes a plurality of insertions and a plurality of deletions, and wherein the plurality of indel pathogenicity scores includes a plurality of insertion scores and a plurality of deletion scores, respectively. In some of such implementations, the applying of the further processed scores at step 812 or the method 800, in general, includes: (1) generating, according to the curve-forming function(s), an insertion curve based on the plurality of insertion scores, (2) generating, according to the curve-forming function(s), a deletion curve based on the plurality of deletion scores, and (3) generating, according to the curve-forming function(s), the missense curve based on the plurality of missense pathogenicity scores.

In some of such implementations, the insertion curve includes a first plurality of data points including an insertion propensity score for each bin of a group of bins. Also, the deletion curve includes a second plurality of data points including a deletion propensity score for each bin of the group of bins. And, the missense curve includes a third plurality of data points including a missense propensity score for each bin of the group of bins. For an example of such data points being displayed on a graph, see FIGS. 12 to 14. In some of such implementations, the insertion propensity score for a bin of the group of bins relates to a proportion of different insertions in the genomes of the population that have insertion scores of the plurality of insertion scores that are associated with the bin. Also, the deletion propensity score for a bin of the group of bins relates to a proportion of different deletions in the genomes of the population that have deletion scores of the plurality of deletion scores that are associated with the bin. And, the missense propensity score for a bin of the group of bins relates to a proportion of variants in the genomes of the population that have missense pathogenicity scores of the plurality of missense pathogenicity scores that are associated with the bin.

With respect to FIG. 9, in some of such implementations, the generating of the insertion curve includes grouping the plurality of insertions into the group of bins. And, it also includes, for each bin of the group of bins: (1) measuring a central tendency distribution of the insertion scores in the bin, and (2) applying the central tendency distribution of the insertion scores in the bin to identify the insertion propensity score for the bin. Also, the generating of the deletion curve includes grouping the plurality of deletions into the group of bins. And, it also includes, for each bin of the group of bins: (1) measuring a central tendency distribution of the deletion scores in the bin, and (2) applying the central tendency distribution of the deletion scores in the bin to identify the deletion propensity score for the bin. Also, the generating of the missense curve includes grouping the plurality of variants into the group of bins. And, it also includes, for each bin of the group of bins: (1) measuring a central tendency distribution of the missense pathogenicity scores in the bin, and (2) applying the central tendency distribution of the missense pathogenicity scores in the bin to identify the insertion propensity score for the bin.

Also, with respect to FIG. 9, in some implementations, the insertion propensity score for a bin of the group of bins represents a probability of one of the plurality of insertions associated with the bin occurs in the genomes of the population given a set of observed insertions. And, the deletion propensity score for the bin represents a probability of one of the plurality of deletions associated with the bin occurs in the genomes of the population given a set of observed deletions. And, the missense propensity score for the bin represents a probability of one of the plurality of variants associated with the bin occurs in the genomes of the population given a set of observed variants. In such examples, the propensity scores reduce selection bias by equating groups based on covariates, and the covariates are the set of observed insertions, the set of observed deletions, and the set of observed variants, respectively.

Also, with respect to FIG. 9, in some implementations, the insertion curve is generated when the first plurality of data points is plotted on a two-dimensional graph with one axis for propensity scores and the other axis for the group of bins. And, the deletion curve is generated when the second plurality of data points is plotted on the two-dimensional graph. And, the missense curve is generated when the third plurality of data points is plotted on the two-dimensional graph. In some of such examples, the two-dimensional graph includes a set of ordered pairs (x, y), wherein f(x)=y, wherein x is the group of bins, and wherein y is the propensity scores. For an example of such data points being displayed on a graph, see FIGS. 12 to 14.

Not depicted in FIG. 9, but inferred from some steps of method 800, in some implementations, the method includes: determining selection pattern differences between the insertion curve and the missense curve, determining one or more second scaling functions to reduce the selection pattern differences between the insertion curve and the missense curve, and enhancing/calibrating/recalibrating/updating/optimizing/modifying the plurality of insertion scores according to the second scaling function(s) to change the output of the ANN. Also, in such implementations, the method 800 includes determining selection pattern differences between the deletion curve and the missense curve, determining one or more third scaling functions to reduce the selection pattern differences between the deletion curve and the missense curve, and enhancing/calibrating/recalibrating/updating/optimizing/modifying the plurality of deletion scores according to the third scaling function(s) to change the output of the ANN. In some implementations, the one or more second scaling functions and the one or more third scaling functions are part of the scaling function(s), and the scaling function(s) include functions to scale the proportions of different insertions, different deletions, and different variants in the genomes of the population, respectively, since indels and single-nucleotide variants have different mutability.

Also, with respect to FIG. 9, in some implementations, the enhancing/calibrating/recalibrating/updating/optimizing/modifying of the plurality of insertion scores includes scaling the insertion propensity scores according to first scaling factors of the scaling factors that are associated with insertions in the genomes of the population. Also, the enhancing/calibrating/recalibrating/updating/optimizing/modifying of the plurality of deletion scores includes scaling the deletion propensity scores according to second scaling factors of the scaling factors that are associated with deletions in the genomes of the population. And, the enhancing/calibrating/recalibrating/updating/optimizing/modifying of the plurality of missense pathogenicity scores includes scaling the missense propensity scores according to third scaling factors of the scaling factors that are associated with variants in the genomes of the population.

Also, with respect to FIG. 9, in some implementations, the scaling function(s) obtain scaling factors from comparable variants under natural selection. See FIG. 11 for an example of results of a function that accounts for natural selection of such variants. In some implementations, comparable variants of the variants are synonymous mutations for variants. In such examples, the enhancing/calibrating/recalibrating/updating/optimizing/modifying of the plurality of missense pathogenicity scores includes calibrating missense propensity scores based on the synonymous mutations for variants. Also, in some implementations, comparable variants of the indels are indels in coding and noncoding regions of the genomes of the population. In such examples, the enhancing/calibrating/recalibrating/updating/optimizing/modifying of the plurality of insertion scores includes calibrating insertion propensity scores based on an observed versus expected ratio based on insertions occurring in coding regions versus noncoding regions of the genomes of the population, respectively. And, the enhancing/calibrating/recalibrating/updating/optimizing/modifying of the plurality of deletion scores includes calibrating deletion propensity scores based on an observed versus expected ratio based on deletions occurring in coding regions versus noncoding regions of the genomes of the population, respectively.

Also, with respect to FIG. 9, in some implementations, the group of bins represents all the scores. Each bin of the group of bins represents a different range of scores in all the scores. And, all the scores includes the plurality of indel pathogenicity scores and the plurality of missense pathogenicity scores. In such examples and others, each bin of the group of bins is associated with a certain amount of the plurality of indels that have scores within a respective range of scores associated with the bin. And, each bin of the group of bins is associated with a certain amount of the plurality of variants that have scores within a respective range of scores associated with the bin.

Also, with respect to FIG. 9, in some implementations, the group of bins includes a group of percentile bins. And, in some examples, the group of percentile bins includes one hundred bins, wherein a first bin of the one hundred bins represents scores that range from 0 to 0.01 and a one hundredth bin represents scores that range from 0.99 to 1, and wherein bins between the first bin and the one hundredth bin each include a range of scores of a percentile.

Also, with respect to FIG. 9, in some implementations, the ANN is configured to classify pathogenicity of variants. In some examples of such implementations, the ANN includes a deep residual neural network for classifying pathogenicity of missense mutations. Even more specific, in some examples, the ANN includes a version of PrimateAI.

Also, with respect to FIG. 9, in some implementations, the first genome database includes a version of a Genome Aggregation Database (gnomAD). In some of such implementations, the second genome database includes a version of the gnomAD. In some instances, the second genome database and the first genome database are the same version of the gnomAD; and in some other implementations, the second and first genome databases are different versions of the gnomAD.

Also, with respect to FIG. 9, in some implementations, measuring the central tendency distribution of the indel pathogenicity scores includes determining a mean of the indel pathogenicity scores. For example, measuring the central tendency distribution of the insertion scores includes determining a mean of the insertion scores and measuring the central tendency distribution of the deletion scores includes determining a mean of the deletion scores. Also, in some implementations, measuring the central tendency distribution of the missense pathogenicity scores includes determining a mean of the missense pathogenicity scores. In some implementations, measuring the central tendency distribution of the indel pathogenicity scores includes determining a mode of the indel pathogenicity scores. For example, measuring the central tendency distribution of the insertion scores includes determining a mode of the insertion scores and measuring the central tendency distribution of the deletion scores includes determining a mode of the deletion scores. Also, in some implementations, measuring the central tendency distribution of the missense pathogenicity scores includes determining a mode of the missense pathogenicity scores. In some implementations, measuring the central tendency distribution of the indel pathogenicity scores includes determining a median of the indel pathogenicity scores. For example, measuring the central tendency distribution of the insertion scores includes determining a median of the insertion scores and measuring the central tendency distribution of the deletion scores includes determining a median of the deletion scores. Also, in some implementations, measuring the central tendency distribution of the missense pathogenicity scores includes determining a median of the missense pathogenicity scores. Also, such techniques apply to even more generic implementations as well. For example, measuring the central tendency distribution of the first-context scores includes determining a mean, mode, or median of the first-context scores. And, measuring the central tendency distribution of the second-context scores includes determining a mean, mode, or median of the second-context scores.

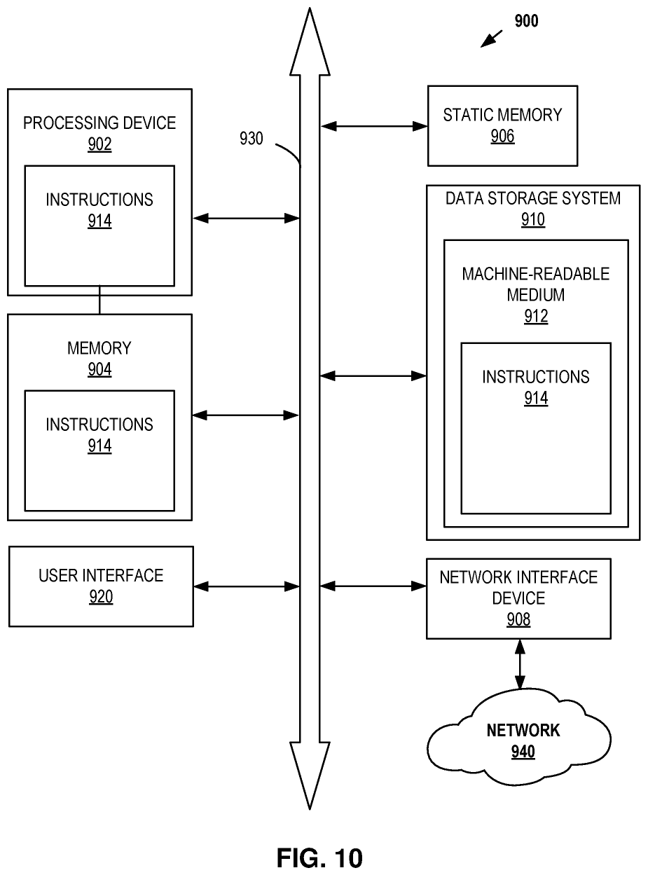

FIG. 10 shows a block diagram of example aspects of the computing system 900, which can include, be or be a part of any one of the electronic or computing systems described herein. FIG. 10 illustrates parts of the computing system 900 within which a set of instructions, for causing the machine to perform any one or more of the methodologies discussed herein, are executed.

In some implementations, the computing system 900 corresponds to a host system that includes, is coupled to, or utilizes memory or is used to perform the operations performed by any one of the computing devices, data processors, and user interface devices described herein. In alternative implementations, the machine is connected (e.g., networked) to other machines in a LAN, an intranet, an extranet, or the Internet. In some implementations, the machine operates in the capacity of a server or a client machine in client-server network environment, as a peer machine in a peer-to-peer (or distributed) network environment, or as a server or a client machine in a cloud computing infrastructure or environment. In some implementations, the machine is a personal computer (PC), a tablet PC, a cellular telephone, a web appliance, a server, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computing system 900 includes a processing device 902, a main memory 904 (e.g., read-only memory (ROM), flash memory, dynamic random-access memory (DRAM), etc.), a static memory 906 (e.g., flash memory, static random-access memory (SRAM), etc.), and a data storage system 910, which communicate with each other via a bus 930. The processing device 902 represents one or more general-purpose processing devices such as a microprocessor, a central processing unit, or the like. More particularly, the processing device is a microprocessor or a processor implementing other instruction sets, or processors implementing a combination of instruction sets. Or, the processing device 902 is one or more special-purpose processing devices such as an application specific integrated circuit (ASIC), a field programmable gate array (FPGA), a digital signal processor (DSP), network processor, or the like. The processing device 902 is configured to execute instructions 914 for performing the operations or steps discussed herein. In some implementations, the computing system 900 includes a network interface device 908 to communicate over a communications network 940 shown in FIG. 10.

The data storage system 910 includes a machine-readable storage medium 912 (also known as a computer-readable medium) on which is stored one or more sets of instructions 914 or software embodying any one or more of the methodologies or functions described herein. The instructions 914 also reside, completely or at least partially, within the main memory 904 or within the processing device 902 during execution thereof by the computing system 900, the main memory 904 and the processing device 902 also constituting machine-readable storage media.

In some implementations, the instructions 914 include instructions to implement functionality corresponding to any one of the computing devices, data processors, user interface devices, and I/O devices described herein. While the machine-readable storage medium 912 is shown in an example implementation to be a single medium, the term “machine-readable storage medium” should be taken to include a single medium or multiple media that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include solid-state memories, optical media, magnetic media, or the like.

Also, as shown, computing system 900 includes user interface 920 that includes a display, in some implementations, and, for example, implements functionality corresponding to any one of the user interface devices disclosed herein. A user interface, such as user interface 920, or a user interface device described herein includes any space or equipment where interactions between humans and machines occur. A user interface described herein allows operation and control of the machine from a human user, while the machine simultaneously provides feedback information to the user. Examples of a user interface (UI), or user interface device include the interactive aspects of computer operating systems (such as graphical user interfaces or GUI), machinery operator controls, and process controls. A UI described herein includes one or more layers, including a human-machine interface (HIM) that interfaces machines with physical input hardware and output hardware.

Also, it is to be understood, that the methodologies discussed herein are computer-implemented methods and, in some implementations, are implementable by the computing system 900. For instance, a computer-implemented method includes an artificial neural network (ANN) generating a plurality of missense pathogenicity scores for each variant of a plurality of variants. Also, the computer-implemented method includes the ANN generating a plurality of indel pathogenicity scores for each indel of a plurality of indels. Further, the computer-implemented method includes applying the plurality of indel pathogenicity scores and the plurality of missense pathogenicity scores to one or more curve-forming functions. And, the computer-implemented method includes applying the further processed scores to the curve-forming function(s) to generate an indel curve and a missense curve and determining selection pattern differences between the indel curve and the missense curve. Also, the computer-implemented method includes determining one or more scaling functions to reduce the selection pattern differences between the curves and updating coefficients of the ANN according to the scaling function(s). The updating the coefficients of the ANN according to the scaling function(s) includes enhancing/calibrating/recalibrating/updating/optimizing/modifying the plurality of indel pathogenicity scores according to the scaling function(s) to provide a recalibrated accuracy of indel pathogenicity score for each indel of the plurality of indels.

FIG. 11 depicts a plot in a two-dimensional graph showing the relationship between binned PrimateAI scores for variants and insertion variants versus natural selection (i.e., propensity of a variant or insertion in genomes of a population). In FIG. 11, natural selection values (or the propensity values) are represented with the y-axis. And, the bins of PrimateAI scores are represented with the x-axis, where the bins are ranges of Primate AI's variant pathogenicity score predictions. The different propensity scores described herein can be or include the natural selection values. The bins of PrimateAI scores can be or include any one of the groups of bins described herein.

FIG. 12 depicts a scatterplot in a two-dimensional graph showing the relationship between binned PrimateAI scores for variants (green points), insertion variants (blue points), and deletion variants (orange points) versus proportions of observed variants (i.e., propensity of a variant or an indel in genomes of a population). In FIG. 12, proportions of observed variants are represented with the y-axis. And, the bins of PrimateAI scores are represented with the x-axis. The different propensity scores described herein can be or include the proportions of observed variants. The bins of PrimateAI scores can be or include any one of the groups of bins described herein.

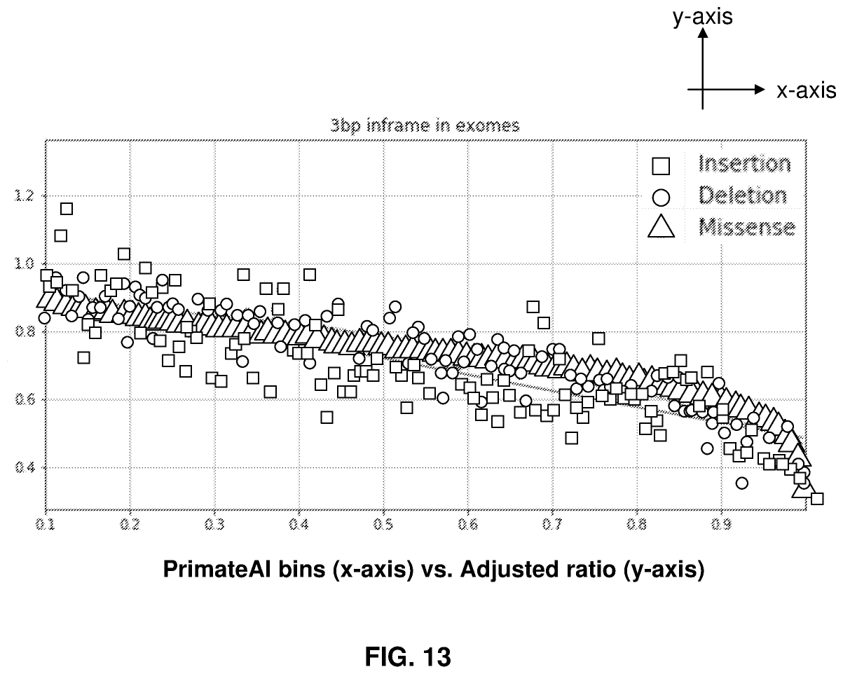

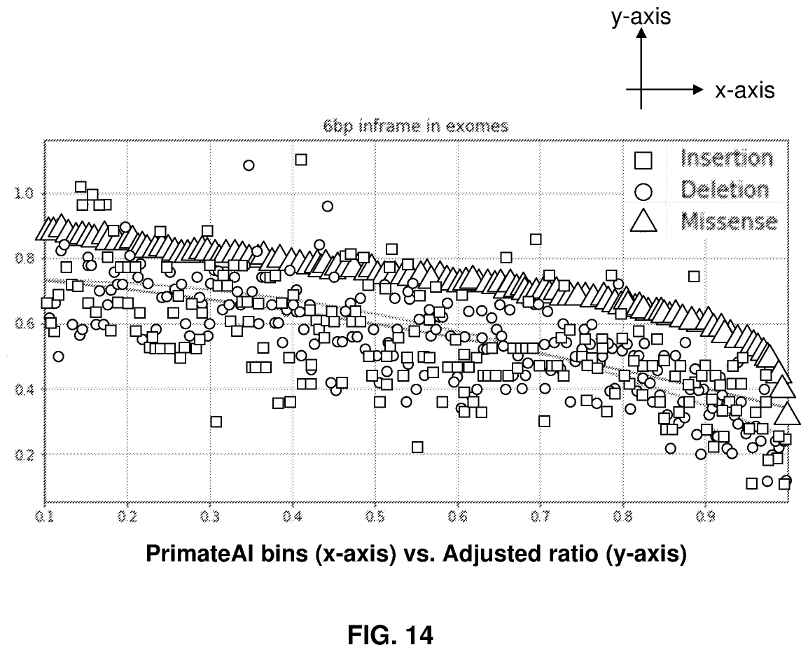

FIGS. 13 and 14 depict respective scatterplots in respective two-dimensional graphs, each plot showing the relationship between binned PrimateAI scores for variants (green points), insertion variants (blue points), and deletion variants (orange points) versus adjusted proportions of observed variants (i.e., propensity of a variant or indel in genomes of a population). In FIGS. 13 and 14, adjusted proportions of observed variants (or the adjusted ratios) are represented with the y-axis. And, the bins of PrimateAI scores are represented with the x-axis. Specifically, FIG. 13 relates to variants occurring in a three base pair in-frame in exomes. Specifically, FIG. 14 relates to variants occurring in a six base pair in-frame in exomes. The different scaled propensity scores described herein can be or include the adjusted proportions of observed variants. The bins of PrimateAI scores can be or include any one of the groups of bins described herein.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a predetermined result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that these and similar terms are to be associated with the appropriate physical quantities and are merely convenient labels applied to these quantities. The present disclosure can refer to the action and processes of a computing system, or similar electronic computing device, which manipulates and transforms data represented as physical (electronic) quantities within the computing system's registers and memories into other data similarly represented as physical quantities within the computing system memories or registers or other such information storage systems.

The present disclosure also relates to an apparatus for performing the operations herein. This apparatus can be specially constructed for the intended purposes, or it can include a general-purpose computer selectively activated or reconfigured by a computer program stored in the computer. Such a computer program can be stored in a computer readable storage medium, such as any type of disk including optical disks, CD-ROMs, and magnetic-optical disks, read-only memories (ROMs), random access memories (RAMs), EPROMs, EEPROMs, magnetic or optical cards, or any type of media suitable for storing electronic instructions, coupled to a computing system bus.

The algorithms and displays presented herein are not inherently related to any particular computer or other apparatus. Various general-purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product, or software, which can include a machine-readable medium having stored thereon instructions, which can be used to program a computing system (or other electronic devices) to perform a process according to the present disclosure. A machine-readable medium includes any mechanism for storing information in a form readable by a machine (e.g., a computer). In some implementations, a machine-readable (e.g., computer-readable) medium includes a machine (e.g., a computer) readable storage medium such as a read only memory (“ROM”), random access memory (“RAM”), magnetic disk storage media, optical storage media, flash memory components, etc.

While the invention has been described in conjunction with the specific implementations described herein, it is evident that many alternatives, combinations, modifications and variations are apparent to those skilled in the art. Accordingly, the example implementations of the invention, as set forth herein are intended to be illustrative only, and not in a limiting sense. Various changes can be made without departing from the spirit and scope of the invention.

We disclose the following clauses:

1. A computer-implemented method, comprising:

processing a plurality of variants to generate a plurality of missense pathogenicity scores for each variant of the plurality of variants;

generating, according to one or more curve-forming functions, a missense curve based on the plurality of missense pathogenicity scores;

processing a plurality of indels to generate a plurality of indel pathogenicity scores for each indel of the plurality of indels;

generating, according to the one or more curve-forming functions, an indel curve based on the plurality of indel pathogenicity scores;

determining selection pattern differences between the indel curve and the missense curve; determining one or more scaling functions to reduce the selection pattern differences between the missense curve and the indel curve; and

enhancing/calibrating/recalibrating/updating/optimizing/modifying the plurality of indel pathogenicity scores according to the one or more scaling functions to provide a recalibrated accuracy of indel pathogenicity score for each indel of the plurality of indels.

2. The computer-implemented method of clause 1, wherein the one or more curve-forming functions comprise a function that accounts for proportions of different indels and proportions of different variants in genomes of a population.

3. The computer-implemented method of clause 2, wherein the one or more curve-forming functions comprise a function that accounts for natural selection of different indels and natural selection of different variants in the genomes of the population.

4. The computer-implemented method of clause 2, wherein the plurality of indels comprises a plurality of insertions and a plurality of deletions, and wherein the plurality of indel pathogenicity scores comprises a plurality of insertion scores and a plurality of deletion scores, respectively.

5. The computer-implemented method of clause 4, comprising:

generating, according to the one or more curve-forming functions, an insertion curve based on the plurality of insertion scores; and

generating, according to the one or more curve-forming functions, a deletion curve based on the plurality of deletion scores.

6. The computer-implemented method of clause 5,

wherein the insertion curve comprises a first plurality of data points comprising an insertion propensity score for each bin of a group of bins,

wherein the deletion curve comprises a second plurality of data points comprising a deletion propensity score for each bin of the group of bins, and

wherein the missense curve comprises a third plurality of data points comprising a missense propensity score for each bin of the group of bins.

7. The computer-implemented method of clause 6,

wherein the insertion propensity score for a bin of the group of bins relates to a proportion of different insertions in the genomes of the population that have insertion scores of the plurality of insertion scores that are associated with the bin,

wherein the deletion propensity score for a bin of the group of bins relates to a proportion of different deletions in the genomes of the population that have deletion scores of the plurality of deletion scores that are associated with the bin, and

wherein the missense propensity score for a bin of the group of bins relates to a proportion of variants in the genomes of the population that have missense pathogenicity scores of the plurality of missense pathogenicity scores that are associated with the bin.

8. The computer-implemented method of clause 7, wherein the generating of the insertion curve comprises:

grouping the plurality of insertions into the group of bins; and

for each bin of the group of bins:
measuring a central tendency distribution of the insertion scores in the bin; and
applying the central tendency distribution of the insertion scores in the bin to identify the insertion propensity score for the bin.

9. The computer-implemented method of clause 7, wherein the generating of the deletion curve comprises:

grouping the plurality of deletions into the group of bins; and

for each bin of the group of bins:
measuring a central tendency distribution of the deletion scores in the bin; and
applying the central tendency distribution of the deletion scores in the bin to identify the deletion propensity score for the bin.

10. The computer-implemented method of clause 7, wherein the generating of the missense curve comprises:

grouping the plurality of variants into the group of bins; and

for each bin of the group of bins:
measuring a central tendency distribution of the missense pathogenicity scores in the bin; and
applying the central tendency distribution of the missense pathogenicity scores in the bin to identify the insertion propensity score for the bin.

11. The computer-implemented method of clause 7,

wherein the insertion propensity score for a bin of the group of bins represents a probability of one of the plurality of insertions associated with the bin occurs in the genomes of the population given a set of observed insertions,

wherein the deletion propensity score for the bin represents a probability of one of the plurality of deletions associated with the bin occurs in the genomes of the population given a set of observed deletions, and

wherein the missense propensity score for the bin represents a probability of one of the plurality of variants associated with the bin occurs in the genomes of the population given a set of observed variants.

12. The computer-implemented method of clause 11, wherein the insertion propensity score, the deletion propensity score, and the missense propensity score reduce selection bias by equating groups based on covariates, and wherein the covariates are the set of observed insertions, the set of observed deletions, and the set of observed variants, respectively.

13. The computer-implemented method of clause 7,

wherein the insertion curve is generated when the first plurality of data points is plotted on a two-dimensional graph with one axis for propensity scores and the other axis for the group of bins,

wherein the deletion curve is generated when the second plurality of data points is plotted on the two-dimensional graph, and

wherein the missense curve is generated when the third plurality of data points is plotted on the two-dimensional graph.

14. The computer-implemented method of clause 13,

wherein the two-dimensional graph comprises a set of ordered pairs (x, y),

wherein f(x)=y,

wherein x is the group of bins, and

wherein y is the propensity scores.

15. The computer-implemented method of clause 13, comprising:

determining selection pattern differences between the insertion curve and the missense curve;

determining one or more second scaling functions to reduce the selection pattern differences between the insertion curve and the missense curve; and

enhancing/calibrating/recalibrating/updating/optimizing/modifying the plurality of insertion scores according to the one or more second scaling functions to provide a recalibrated accuracy of insertion pathogenicity score for each insertion of the plurality of insertions.

16. The computer-implemented method of clause 15, comprising:

determining selection pattern differences between the deletion curve and the missense curve;

determining one or more third scaling functions to reduce the selection pattern differences between the deletion curve and the missense curve; and

enhancing/calibrating/recalibrating/updating/optimizing/modifying the plurality of deletion scores according to the one or more third scaling functions to provide a recalibrated accuracy of deletion pathogenicity score for each deletion of the plurality of deletions.

17. The computer-implemented method of clause 16,

wherein the one or more second scaling functions and the one or more third scaling functions are part of the one or more scaling functions, and

wherein the one or more scaling functions comprise functions to scale the proportions of different insertions, different deletions, and different variants in the genomes of the population, respectively, since indels and single-nucleotide variants have different mutability.

18. The computer-implemented method of clause 17, wherein the one or more scaling functions obtain scaling factors from comparable variants under natural selection.

19. The computer-implemented method of clause 18,

wherein the enhancing/calibrating/recalibrating/updating/optimizing/modifying of the plurality of insertion scores comprises scaling a plurality of insertion propensity scores according to first scaling factors of the scaling factors that are associated with insertions in the genomes of the population,

wherein the enhancing/calibrating/recalibrating/updating/optimizing/modifying of the plurality of deletion scores comprises scaling a plurality of deletion propensity scores according to second scaling factors of the scaling factors that are associated with deletions in the genomes of the population, and

wherein the enhancing/calibrating/recalibrating/updating/optimizing/modifying of the plurality of missense pathogenicity scores comprises scaling a plurality of missense propensity scores according to third scaling factors of the scaling factors that are associated with variants in the genomes of the population.

20. The computer-implemented method of clause 19, wherein comparable variants of the variants are synonymous mutations for variants.

21. The computer-implemented method of clause 20, wherein the enhancing/calibrating/recalibrating/updating/optimizing/modifying of the plurality of missense pathogenicity scores comprises calibrating missense propensity scores based on the synonymous mutations for variants.

22. The computer-implemented method of clause 19, wherein the comparable variants of the indels are indels in coding and noncoding regions of the genomes of the population.

23. The computer-implemented method of clause 22,

wherein the enhancing/calibrating/recalibrating/updating/optimizing/modifying of the plurality of insertion scores comprises calibrating insertion propensity scores based on an observed versus expected ratio based on insertions occurring in coding regions versus noncoding regions of the genomes of the population, respectively, and

wherein the enhancing/calibrating/recalibrating/updating/optimizing/modifying of the plurality of deletion scores comprises calibrating deletion propensity scores based on an observed versus expected ratio based on deletions occurring in coding regions versus noncoding regions of the genomes of the population, respectively.

24. The computer-implemented method of clause 6,

wherein the group of bins represents all scores,

wherein each bin of the group of bins represents a different range of scores in all the scores, and

wherein all the scores comprise the plurality of insertion scores, the plurality of deletion scores, and the plurality of missense pathogenicity scores.

25. The computer-implemented method of clause 24,

wherein each bin of the group of bins is associated with a certain amount of the plurality of insertions that have scores within a respective range of scores associated with the bin,

wherein each bin of the group of bins is associated with a certain amount of the plurality of deletions that have scores within a respective range of scores associated with the bin, and

wherein each bin of the group of bins is associated with a certain amount of the plurality of variants that have scores within a respective range of scores associated with the bin.

26. The computer-implemented method of clause 25, wherein the group of bins comprises a group of percentile bins.

27. The computer-implemented method of clause 26, wherein the group of percentile bins comprises one hundred bins, wherein a first bin of the one hundred bins represents scores that range from 0 to 0.01 and a one hundredth bin represents scores that range from 0.99 to 1, and wherein bins between the first bin and the one hundredth bin each comprise a range of scores of a percentile.

28. The computer-implemented method of clause 1, wherein the plurality of indel pathogenicity scores is generated by an artificial neural network (ANN), and wherein the processing of the plurality of indels is implemented by the ANN.

29. The computer-implemented method of clause 28, wherein the ANN is configured to classify pathogenicity of variants.

30. The computer-implemented method of clause 29, wherein the ANN comprises a deep residual neural network for classifying pathogenicity of missense mutations.

31. The computer-implemented method of clause 30, wherein the ANN comprises a version of PrimateAI.

32. The computer-implemented method of clause 1, further comprising identifying the plurality of variants in a first genome database.

33. The computer-implemented method of clause 32, wherein the first genome database comprises a version of a Genome Aggregation Database (gnomAD).

34. The computer-implemented method of clause 32, further comprising identifying the plurality of indels in a second genome database.

35. The computer-implemented method of clause 34, wherein the second genome database comprises a version of the gnomAD.

36. The computer-implemented method of clause 1, further comprising:

identifying the plurality of variants in a first genome database; and

identifying the plurality of indels in a second genome database,
wherein the first and the second genome databases are parts of one or more versions of a Genome Aggregation Database (gnomAD).

37. The computer-implemented method of clause 1, wherein the generating of the indel curve comprises grouping the plurality of indels into a group of bins.

38. The computer-implemented method of clause 37, wherein the generating of the missense curve comprises grouping the plurality of variants into the group of bins.

39. The computer-implemented method of clause 38, wherein the generating of the indel curve comprises, for each bin of the group of bins:

measuring a central tendency distribution of indel pathogenicity scores in the bin; and

applying the central tendency distribution of the indel pathogenicity scores in the bin to identify an indel propensity score for the bin.

40. The computer-implemented method of clause 39, wherein the generating of the missense curve comprises, for each bin of the group of bins:

measuring a central tendency distribution of missense pathogenicity scores in the bin; and

applying the central tendency distribution of the missense pathogenicity scores in the bin to identify a missense propensity score for the bin.

41. The computer-implemented method of clause 39, wherein measuring the central tendency distribution of the indel pathogenicity scores comprises determining a mean of the indel pathogenicity scores.

42. The computer-implemented method of clause 40, wherein measuring the central tendency distribution of the missense pathogenicity scores comprises determining a mean of the missense pathogenicity scores.

43. The computer-implemented method of clause 39, wherein measuring the central tendency distribution of the indel pathogenicity scores comprises determining a median of the indel pathogenicity scores.

44. The computer-implemented method of clause 40, wherein measuring the central tendency distribution of the missense pathogenicity scores comprises determining a median of the indel pathogenicity scores.

45. The computer-implemented method of clause 39, wherein measuring the central tendency distribution of the indel pathogenicity scores comprises determining a mode of the indel pathogenicity scores.

46. The computer-implemented method of clause 40, wherein measuring the central tendency distribution of the missense pathogenicity scores comprises determining a mode of the indel pathogenicity scores.

47. The computer-implemented method of clause 8, wherein measuring the central tendency distribution of the insertion scores comprises determining a mean of the insertion scores.

48. The computer-implemented method of clause 9, wherein measuring the central tendency distribution of the deletion scores comprises determining a mean of the deletion scores.

49. The computer-implemented method of clause 10, wherein measuring the central tendency distribution of the missense pathogenicity scores comprises determining a mean of the missense pathogenicity scores.

50. The computer-implemented method of clause 10, wherein measuring the central tendencies of the insertion scores, the deletion scores, and the missense pathogenicity scores comprises determining a mode or a median of the scores.

51. A computer-implemented method, comprising:

identifying a plurality of variants in a first genome database;

identifying a plurality of indels in a second genome database;

generating, by an artificial neural network (ANN), a plurality of missense pathogenicity scores for each variant of the plurality of variants;

generating, by the ANN, a plurality of indel pathogenicity scores for each indel of the plurality of indels;

applying the plurality of indel pathogenicity scores and the plurality of missense pathogenicity scores to one or more curve-forming functions;

further processing the plurality of missense pathogenicity scores and the plurality of indel pathogenicity scores using the one or more curve-forming functions to generate an indel curve and a missense curve;

determining selection pattern differences between the indel curve and the missense curve; determining one or more scaling functions to reduce the selection pattern differences between the indel curve and the missense curve; and

updating coefficients of the ANN according to the one or more scaling functions.

52. The computer-implemented method of clause 51,

wherein further processing the plurality of indel pathogenicity scores and the plurality of missense pathogenicity scores using the one or more curve-forming functions comprises:
grouping the plurality of variants into a group of bins;
grouping the plurality of indels into the group of bins; and
for each bin of the group of bins:
   measuring a central tendency distribution of indel pathogenicity scores in the bin; and
   measuring a central tendency distribution of missense pathogenicity scores in the bin; and
applying the central tendencies of the indel pathogenicity scores and the missense pathogenicity scores to the one or more curve-forming functions to generate the indel curve and the missense curve.

53. The computer-implemented method of clause 51, wherein the one or more curve-forming functions comprise a function that accounts for proportions of different indels and proportions of different variants in genomes of a population.

54. The computer-implemented method of clause 53, wherein the one or more curve-forming functions comprise a function that accounts for natural selection of different indels and natural selection of different variants in the genomes of the population.

55. The computer-implemented method of clause 53, wherein the plurality of indels comprises a plurality of insertions and a plurality of deletions, and wherein the plurality of indel pathogenicity scores comprises a plurality of insertion scores and a plurality of deletion scores, respectively.

56. The computer-implemented method of clause 55, comprising:

generating, according to the one or more curve-forming functions, an insertion curve based on the plurality of insertion scores;

generating, according to the one or more curve-forming functions, a deletion curve based on the plurality of deletion scores; and

generating, according to the one or more curve-forming functions, the missense curve based on the plurality of missense pathogenicity scores.

57. The computer-implemented method of clause 56,

wherein the insertion curve comprises a first plurality of data points comprising an insertion propensity score for each bin of a group of bins,

wherein the deletion curve comprises a second plurality of data points comprising a deletion propensity score for each bin of the group of bins, and

wherein the missense curve comprises a third plurality of data points comprising a missense propensity score for each bin of the group of bins.

58. The computer-implemented method of clause 57,

wherein the insertion propensity score for a bin of the group of bins relates to a proportion of different insertions in the genomes of the population that have insertion scores of the plurality of insertion scores that are associated with the bin,

wherein the deletion propensity score for a bin of the group of bins relates to a proportion of different deletions in the genomes of the population that have deletion scores of the plurality of deletion scores that are associated with the bin, and

wherein the missense propensity score for a bin of the group of bins relates to a proportion of variants in the genomes of the population that have missense pathogenicity scores of the plurality of missense pathogenicity scores that are associated with the bin.

59. The computer-implemented method of clause 58, wherein the generating of the insertion curve comprises:

grouping the plurality of insertions into the group of bins; and

for each bin of the group of bins:
measuring a central tendency distribution of the insertion scores in the bin; and
applying the central tendency distribution of the insertion scores in the bin to identify the insertion propensity score for the bin.

60. The computer-implemented method of clause 58, wherein the generating of the deletion curve comprises:

grouping the plurality of deletions into the group of bins; and

for each bin of the group of bins:
measuring a central tendency distribution of the deletion scores in the bin; and
applying the central tendency distribution of the deletion scores in the bin to identify the deletion propensity score for the bin.

61. The computer-implemented method of clause 58, wherein the generating of the missense curve comprises:

grouping the plurality of variants into the group of bins; and

for each bin of the group of bins:
measuring a central tendency distribution of missense pathogenicity scores in the bin; and
applying the central tendency distribution of the missense pathogenicity scores in the bin to identify the insertion propensity score for the bin.

62. The computer-implemented method of clause 58,

wherein the insertion propensity score for a bin of the group of bins represents a probability of one of the plurality of insertions associated with the bin occurs in the genomes of the population given a set of observed insertions,

wherein the deletion propensity score for the bin represents a probability of one of the plurality of deletions associated with the bin occurs in the genomes of the population given a set of observed deletions, and

wherein the missense propensity score for the bin represents a probability of one of the plurality of variants associated with the bin occurs in the genomes of the population given a set of observed variants.

63. The computer-implemented method of clause 62, wherein the insertion propensity score, the deletion propensity score, and the missense propensity score reduce selection bias by equating groups based on covariates, and wherein the covariates are the set of observed insertions, the set of observed deletions, and the set of observed variants, respectively.

64. The computer-implemented method of clause 58,

wherein the insertion curve is generated when the first plurality of data points is plotted on a two-dimensional graph with one axis for propensity scores and the other axis for the group of bins,

wherein the deletion curve is generated when the second plurality of data points is plotted on the two-dimensional graph, and

wherein the missense curve is generated when the third plurality of data points is plotted on the two-dimensional graph.

65. The computer-implemented method of clause 64,

wherein the two-dimensional graph comprises a set of ordered pairs (x, y),

wherein f(x)=y,

wherein x is the group of bins, and

wherein y is the propensity scores.

66. The computer-implemented method of clause 64, comprising:

determining selection pattern differences between the insertion curve and the missense curve;

determining one or more second scaling functions to reduce the selection pattern differences between the insertion curve and the missense curve; and

enhancing/calibrating/recalibrating/updating/optimizing/modifying the plurality of insertion scores according to the one or more second scaling functions to change the output of the ANN.

67. The computer-implemented method of clause 66, comprising:

determining selection pattern differences between the deletion curve and the missense curve;

determining one or more third scaling functions to reduce the selection pattern differences between the deletion curve and the missense curve; and

enhancing/calibrating/recalibrating/updating/optimizing/modifying the plurality of deletion scores according to the one or more third scaling functions to change the output of the ANN.

68. The computer-implemented method of clause 67,

wherein the one or more second scaling functions and the one or more third scaling functions are part of the one or more scaling functions, and

wherein the one or more scaling functions comprise functions to scale the proportions of different insertions, different deletions, and different variants in the genomes of the population, respectively, since indels and single-nucleotide variants have different mutability.

69. The computer-implemented method of clause 68, wherein the one or more scaling functions obtain scaling factors from comparable variants under natural selection.

70. The computer-implemented method of clause 69,

wherein the enhancing/calibrating/recalibrating/updating/optimizing/modifying of the plurality of insertion scores comprises scaling the plurality of insertion propensity scores according to first scaling factors of the scaling factors that are associated with insertions in the genomes of the population,

wherein the enhancing/calibrating/recalibrating/updating/optimizing/modifying of the plurality of deletion scores comprises scaling the plurality of deletion propensity scores according to second scaling factors of the scaling factors that are associated with deletions in the genomes of the population, and

wherein the enhancing/calibrating/recalibrating/updating/optimizing/modifying of the plurality of missense pathogenicity scores comprises scaling the plurality of missense propensity scores according to third scaling factors of the scaling factors that are associated with variants in the genomes of the population.

71. The computer-implemented method of clause 68, wherein comparable variants of the variants are synonymous mutations for variants.

72. The computer-implemented method of clause 71, wherein the enhancing/calibrating/recalibrating/updating/optimizing/modifying of the plurality of missense pathogenicity scores comprises calibrating missense propensity scores based on the synonymous mutations for variants.

73. The computer-implemented method of clause 72, wherein comparable variants of the indels are indels in coding and noncoding regions of the genomes of the population.

74. The computer-implemented method of clause 73,

wherein the enhancing/calibrating/recalibrating/updating/optimizing/modifying of the plurality of insertion scores comprises calibrating insertion propensity scores based on an observed versus expected ratio based on insertions occurring in coding regions versus noncoding regions of the genomes of the population, respectively, and

wherein the enhancing/calibrating/recalibrating/updating/optimizing/modifying of the plurality of deletion scores comprises calibrating deletion propensity scores based on an observed versus expected ratio based on deletions occurring in coding regions versus noncoding regions of the genomes of the population, respectively.

75. The computer-implemented method of clause 52,

wherein the group of bins represents all scores,

wherein each bin of the group of bins represents a different range of scores in all the scores, and

wherein all the scores comprise the plurality of indel pathogenicity scores and the plurality of missense pathogenicity scores.

76. The computer-implemented method of clause 75,

wherein each bin of the group of bins is associated with a certain amount of the plurality of indels that have scores within a respective range of scores associated with the bin, and

wherein each bin of the group of bins is associated with a certain amount of the plurality of variants that have scores within a respective range of scores associated with the bin.

77. The computer-implemented method of clause 76, wherein the group of bins comprises a group of percentile bins.

78. The computer-implemented method of clause 77, wherein the group of percentile bins comprises one hundred bins, wherein a first bin of the one hundred bins represents scores that range from 0 to 0.01 and a one hundredth bin represents scores that range from 0.99 to 1, and wherein bins between the first bin and the one hundredth bin each comprise a range of scores of a percentile.

79. The computer-implemented method of clause 51, wherein the ANN is configured to classify pathogenicity of variants.

80. The computer-implemented method of clause 79, wherein the ANN comprises a deep residual neural network for classifying pathogenicity of missense mutations.

81. The computer-implemented method of clause 80, wherein the ANN comprises a version of PrimateAI.

82. The computer-implemented method of clause 51, wherein the first genome database comprises a version of a Genome Aggregation Database (gnomAD).

83. The computer-implemented method of clause 82, wherein the second genome database comprises a version of the gnomAD.

84. The computer-implemented method of clause 52, wherein measuring the central tendency distribution of the indel pathogenicity scores in the bin comprises determining a mean of the indel pathogenicity scores.

85. The computer-implemented method of clause 52, wherein measuring the central tendency distribution of the missense pathogenicity scores in the bin comprises determining a mean of the missense pathogenicity scores.

86. The computer-implemented method of clause 52, wherein measuring the central tendencies of the indel pathogenicity scores and the missense pathogenicity scores in the bin comprises determining a mode or a median of the scores.

87. The computer-implemented method of clause 59, wherein measuring the central tendency distribution of the insertion scores comprises determining a mean of the insertion scores.

88. The computer-implemented method of clause 60, wherein measuring the central tendency distribution of the deletion scores comprises determining a mean of the deletion scores.

89. The computer-implemented method of clause 61, wherein measuring the central tendency distribution of the missense pathogenicity scores in the bin comprises determining a mean of the missense pathogenicity scores.

90. The computer-implemented method of clause 59, wherein measuring the central tendency distribution of the insertion scores comprises determining a mode or a median of the insertion scores.

91. The computer-implemented method of clause 60, wherein measuring the central tendency distribution of the deletion scores comprises determining a mode or a median of the deletion scores.

92. The computer-implemented method of clause 61, wherein measuring the central tendency distribution of the missense pathogenicity scores in the bin comprises determining a mode or a median of the missense pathogenicity scores.

93. The computer-implemented method of clause 51, wherein the updating the coefficients of the ANN according to the one or more scaling functions comprises enhancing/calibrating/recalibrating/updating/optimizing/modifying the plurality of indel pathogenicity scores according to the one or more scaling functions to provide a recalibrated accuracy of indel pathogenicity score for each indel of the plurality of indels.

94. A computer-implemented method, comprising:

generating, by an artificial neural network (ANN), a plurality of missense pathogenicity scores for each variant of a plurality of variants;

generating, by the ANN, a plurality of indel pathogenicity scores for each indel of a plurality of indels;

applying the plurality of indel pathogenicity scores and the plurality of missense pathogenicity scores to one or more curve-forming functions;

further processing the plurality of indel pathogenicity scores and the plurality of missense pathogenicity scores using the one or more curve-forming functions to generate an indel curve and a missense curve;

determining selection pattern differences between the indel curve and the missense curve;

determining one or more scaling functions to reduce the selection pattern differences between the indel curve and the missense curve; and

updating coefficients of the ANN according to the one or more scaling functions, and
wherein the updating the coefficients of the ANN according to the one or more scaling functions comprises enhancing/calibrating/recalibrating/updating/optimizing/modifying the plurality of indel pathogenicity scores according to the one or more scaling functions to provide a recalibrated accuracy of indel pathogenicity score for each indel of the plurality of indels.

What we claim is:

1. A system comprising:
at least one processor; and
a non-transitory computer readable medium comprising instructions that, when executed by the at least one processor, cause the system to:
process a plurality of variants to generate a plurality of missense pathogenicity scores for each variant of the plurality of variants;
generate, according to one or more curve-forming functions, a missense curve based on the plurality of missense pathogenicity scores;
process a plurality of indels to generate a plurality of indel pathogenicity scores for each indel of the plurality of indels;
generate, according to the one or more curve-forming functions, an indel curve based on the plurality of indel pathogenicity scores;
determine selection pattern differences between the indel curve and the missense curve;
determine one or more scaling functions to reduce the selection pattern differences between the missense curve and the indel curve; and
modify the plurality of indel pathogenicity scores according to the one or more scaling functions to provide a recalibrated accuracy of indel pathogenicity score for each indel of the plurality of indels.

2. The system of claim 1, wherein the one or more curve-forming functions comprise a function that accounts for proportions of different indels and proportions of different variants in genomes of a population.

3. The system of claim 2, wherein the one or more curve-forming functions comprise a function that accounts for natural selection of different indels and natural selection of different variants in the genomes of the population.

4. The system of claim 2, wherein the plurality of indels comprises a plurality of insertions and a plurality of deletions, and wherein the plurality of indel pathogenicity scores comprises a plurality of insertion scores and a plurality of deletion scores, respectively.

5. The system of claim 4, further comprising instructions that, when executed by the at least one processor, cause the system to:
generate, according to the one or more curve-forming functions, an insertion curve based on the plurality of insertion scores; and
generate, according to the one or more curve-forming functions, a deletion curve based on the plurality of deletion scores.

6. The system of claim 5,
wherein the insertion curve comprises a first plurality of data points comprising an insertion propensity score for each bin of a group of bins,
wherein the deletion curve comprises a second plurality of data points comprising a deletion propensity score for each bin of the group of bins, and
wherein the missense curve comprises a third plurality of data points comprising a missense propensity score for each bin of the group of bins.

7. The system of claim 5, further comprising instructions that, when executed by the at least one processor, cause the system to:
determine selection pattern differences between the insertion curve and the missense curve;
determine one or more second scaling functions to reduce the selection pattern differences between the insertion curve and the missense curve; and
modify the plurality of insertion scores according to the one or more second scaling functions to provide a recalibrated accuracy of insertion pathogenicity score for each insertion of the plurality of insertions.

8. The system of claim 1, further comprising instructions that, when executed by the at least one processor, cause the system to generate the plurality of indel pathogenicity scores by utilizing an artificial neural network (ANN) to process the plurality of indels and generate the plurality of indel pathogenicity scores.

9. The system of claim 1, further comprising instructions that, when executed by the at least one processor, cause the system to:
identify the plurality of variants in a first genome database; and
identify the plurality of indels in a second genome database.

10. A computer-implemented method comprising:
processing a plurality of variants to generate a plurality of missense pathogenicity scores for each variant of the plurality of variants;
generating, according to one or more curve-forming functions, a missense curve based on the plurality of missense pathogenicity scores;
processing a plurality of indels to generate a plurality of indel pathogenicity scores for each indel of the plurality of indels;
generating, according to the one or more curve-forming functions, an indel curve based on the plurality of indel pathogenicity scores;
determining selection pattern differences between the indel curve and the missense curve;
determining one or more scaling functions to reduce the selection pattern differences between the missense curve and the indel curve; and
modifying the plurality of indel pathogenicity scores according to the one or more scaling functions to provide a recalibrated accuracy of indel pathogenicity score for each indel of the plurality of indels.

11. The computer-implemented method of claim 10, wherein the one or more curve-forming functions comprise a function that accounts for proportions of different indels and proportions of different variants in genomes of a population.

12. The computer-implemented method of claim 11, wherein the one or more curve-forming functions comprise a function that accounts for natural selection of different indels and natural selection of different variants in the genomes of the population.

13. The computer-implemented method of claim 11, wherein the plurality of indels comprises a plurality of insertions and a plurality of deletions, and wherein the plurality of indel pathogenicity scores comprises a plurality of insertion scores and a plurality of deletion scores, respectively.

14. The computer-implemented method of claim 13, further comprising:
generating, according to the one or more curve-forming functions, an insertion curve based on the plurality of insertion scores; and
generating, according to the one or more curve-forming functions, a deletion curve based on the plurality of deletion scores.

15. The computer-implemented method of claim 14,
wherein the insertion curve comprises a first plurality of data points comprising an insertion propensity score for each bin of a group of bins,
wherein the deletion curve comprises a second plurality of data points comprising a deletion propensity score for each bin of the group of bins, and
wherein the missense curve comprises a third plurality of data points comprising a missense propensity score for each bin of the group of bins.

16. The computer-implemented method of claim 15, further comprising:
determining selection pattern differences between the insertion curve and the missense curve;
determining one or more second scaling functions to reduce the selection pattern differences between the insertion curve and the missense curve; and
modifying the plurality of insertion scores according to the one or more second scaling functions to provide a recalibrated accuracy of insertion pathogenicity score for each insertion of the plurality of insertions.

17. The computer-implemented method of claim 10, wherein the plurality of indel pathogenicity scores is generated by an artificial neural network (ANN), and wherein the processing of the plurality of indels is implemented by the ANN, and wherein the ANN is configured to classify pathogenicity of variants.

18. The computer-implemented method of claim 10, further comprising:
identifying the plurality of variants in a first genome database; and
identifying the plurality of indels in a second genome database.

19. A non-transitory computer-readable medium storing instructions that, when executed by at least one processor, cause a computing device to:
process a plurality of variants to generate a plurality of missense pathogenicity scores for each variant of the plurality of variants;
generate, according to one or more curve-forming functions, a missense curve based on the plurality of missense pathogenicity scores;
process a plurality of indels to generate a plurality of indel pathogenicity scores for each indel of the plurality of indels;
generate, according to the one or more curve-forming functions, an indel curve based on the plurality of indel pathogenicity scores;
determine selection pattern differences between the indel curve and the missense curve;
determine one or more scaling functions to reduce the selection pattern differences between the missense curve and the indel curve; and
modify the plurality of indel pathogenicity scores according to the one or more scaling functions to provide a recalibrated accuracy of indel pathogenicity score for each indel of the plurality of indels.

20. The non-transitory computer-readable medium of claim 19, further storing instructions that, when executed by the at least one processor, cause the computing device to generate the plurality of indel pathogenicity scores by utilizing an artificial neural network (ANN) to process the plurality of indels and generate the plurality of indel pathogenicity scores.
